(19) United States
(12) Patent Application Publication
(10) Pub. No.: US 2026/0157964 A1
(43) Pub. Date: Jun. 11, 2026

(54) LIPOPHILIC STATIN COMPOSITION WITH IMPROVED SOLUBILITY AND PERMEABILITY AND USES THEREOF

(71) Applicants: KOREA INSTITUTE OF SCIENCE AND TECHNOLOGY, Seoul (KR); MOKPO NATIONAL UNIVERSITY INDUSTRY-ACADEMIA COOPERATION GROUP, Jeollanam-do (KR)

(72) Inventors: In-San KIM, Seoul (KR); Jin Woo PARK, Seoul (KR); Gi-hoon NAM, Seoul (KR); Seong A KIM, Seoul (KR); Youngrang BAE, Seoul (KR)

(73) Assignee: KOREA INSTITUTE OF SCIENCE AND TECHNOLOGY, Seoul (KR)

(21) Appl. No.: 18/550,892

(22) PCT Filed: Jun. 2, 2021
(86) PCT No.: PCT/KR2021/006868

§ 371 Date: Sep. 15, 2023

(30) Foreign Application Priority Data
Mar. 17, 2021 (KR) 10-2021-0034814

(51) Int. Cl. A61K 9/107; A61K 9/00; A61K 31/366; A61K 31/402; A61K 31/405; A61K 31/47; A61K 45/06; A61K 47/28; A61P 35/00

(52) U.S. Cl. CPC A61K 9/1075; A61K 9/0051; A61K 31/366; A61K 31/402; A61K 31/405; A61K 31/47; A61K 45/06; A61K 47/28; A61P 35/00

(57) ABSTRACT

Provided are a lipophilic statin composition with improved solubility and permeability and uses thereof. The simvastatin (SIMVA)/DL-colloidal dispersion (CD) composite according to the present invention may be applied to effective anticancer immunotherapy.

### TECHNICAL FIELD

The present invention relates to a lipophilic statin composition with improved solubility and permeability and uses thereof.

### BACKGROUND ART

Statins are compounds known for their anticancer effects. Specifically, statins are compounds suppressing 3-hydroxy-3-methylglutaryl-coenzyme A reductase, which regulates the first step of a mevalonate pathway involved in synthesis of cholesterol and isoprenoid lipids. The isoprenoid lipids (e.g., farnesyl or geranylgeranyl) induce prenylation of proteins, and prenylated proteins are immobilized on the inner lobules of cell membranes to initiate various signaling pathways involved in oncogenesis, including mitogen-activated protein kinase, p53, and phosphoinositide 3-kinase pathways.

Simvastatin (SIMVA), as a highly lipophilic lipid-lowering prodrug extracted from statins, has been known for its superior antitumor effects on hydrophilic statins not only in cohort studies but also under a variety of in vitro and in vivo experimental conditions. Also, in a variety of studies, antitumor effects of SIMVA against various types of cancer mediated by apoptosis, necrotic cell death, and induction of chemotherapy drug sensitization were proved. In particular, it has been proved that SIMVA accelerates the cancer immunity cycle by inducing continuous tumor-specific immunity. For example, SIMVA induced a decrease in tumorigenic tumor-associated macrophages (TAMs) in premalignant human lung adenocarcinoma in a dose-dependent manner. Also, SIMVA has been used as a cancer vaccine adjuvant to enhance antigen presentation and, in consideration of the fact that a high cholesterol level in the tumor microenvironment (TME) is related to depletion of tumor-infiltrating CD8⁺ T cells that express immune checkpoint, a decreased cholesterol level of CD8⁺ T cells by SIMVA may recover T cell-mediated antitumor activity. These results indicate the potential of SIMVA in anticancer immunotherapy.

Although anticancer effects of SIMVA have been confirmed as described above, it is difficult to apply SIMVA to clinical trials since an optimal dose of SIMVA and a treatment schedule thereof have not yet been established. Furthermore, since lipophilic SIMVA is poorly water-soluble and undergoes various absorption processes and extensive first pass metabolism via cytochrome 3A (CYP3A) in the intestines and liver, resulting in a low in vivo oral bioavailability of approximately 5%, several studies have suggested relatively high statin doses for treating a cancer which sometimes exceed a dose required for treating hypercholesterolemia by 100 times. However, plasma statin concentrations cannot be increased by simply increasing the dose of statin. Although SIMVA is known to be tolerated at a dose of 80 mg or less and to be safer than other statins, it has been reported that high-dose SIMVA treatment temporarily stimulates osteoclast activity and causes gastrointestinal side effects in pretreated multiple myeloma patients, and serious side effects such as myopathy and rhabdomyolysis may be caused by high-dose SIMVA treatment. Therefore, continuous efforts, including development of solid dispersions, nanostructured lipid carriers, nanoparticles, nanoemulsion particles, and self-nanoemulsifying formulations, have been made to improve in vivo oral bioavailability.

### DISCLOSURE OF INVENTION

### Technical Problem

As a result of improving physicochemical properties such as solubility and permeability and in vivo oral bioavailability of SIMVA by developing an oral delivery system of SIMVA based on a colloidal dispersion (CD), the present inventors have found that cellular uptake of SIMVA is increased, and accordingly, antitumor effects and immunoregulatory effects are considerably improved, thereby completing the present invention.

### Solution to Problem

An object of the present invention is to provide a composition for preventing or treating a cancer including: i) a lipophilic statin; ii) a composite of deoxycholic acid (DOCA) or a pharmaceutically acceptable salt thereof and an amino acid or an acceptable salt thereof; and iii) a surfactant.

Another object of the present invention is to provide a method of preparing a composition including: i) a lipophilic statin; ii) a composite of deoxycholic acid (DOCA) or a pharmaceutically acceptable salt thereof and an amino acid or an acceptable salt thereof; and iii) a surfactant.

Another object of the present invention is to provide a method of preventing or treating a cancer including administering the composition to an individual.

Another object of the present invention is to provide a use of a composition for preventing or treating a cancer, comprising: i) a lipophilic statin; ii) a composite of deoxycholic acid (DOCA) or a pharmaceutically acceptable salt thereof and an amino acid or an acceptable salt thereof; and iii) a surfactant.

### Advantageous Effects of Invention

According to the present invention, solubility and permeability of the lipophilic statin were considerably improved using the lipophilic statin/DL-CD composite for oral administration prepared using N^{α}-deoxycholyl-I-lysyl-methylester (DL), as an oral permeation enhancer, and Labrasol and Poloxamer 188 (P188), as surfactants, specifically the simvastatin (SIMVA)/DL-colloidal dispersion (CD) composite. In addition, oral absorption of the SIMVA/DL-CD composite was considerably increased, and in vivo experiments for CT26 tumor-bearing mice administered with the SIMVA/DL-CD composite exhibited considerable tumor growth suppressing effects via elevated anticancer immunity. In particular, the co-administration of the SIMVA/DL-CD composite for oral administration and OP powder induced considerable tumor suppressing effects and CD8⁺ T cell immunity. In addition, the co-administration sensitized tumors resistant to αPD-1 monoclonal antibody to checkpoint blockade, and thus the lipophilic statin/DL-CD composite according to the present invention may be applied to effective anticancer immunotherapy.

### BRIEF DESCRIPTION OF DRAWINGS

FIG. 1 shows simvastatin (SIMVA) present in an amorphous form in a solid dispersion of N_{α}-deoxycholyl-I-lysyl-methylester (DL), Poloxamer 188 (P188), and Labrasol, which is a colloidal dispersion (CD) of an aqueous phase. (A) Powder X-ray diffraction pattern, (B) Differential scanning calorimetric thermograms of pure SIMVA, DL, P188, SIMVA/DL, SIMVA/P188, SIMVA-CD, SIMVA/DL-CD, and a physical mixture of SIMVA, DL, and P188, (C) Schematic diagram of a structure of SIMVA/DL-CD. (C) is a schematic diagram of a self-assembled micelle in an aqueous phase. (D) TEM images of SIMVA/DL-CD. Scale bars indicate 200 nm and 100 nm, respectively.

FIG. 2 shows in vitro cytotoxicity of SIMVA in 5% dimethyl sulfoxide (SIMVA in 5% DMSO, N^{α}-deoxycholyl-I-lysyl-methylester (DL), a colloidal dispersion of Labrasol and Poloxamer 188 (P188) (colloidal dispersion, CD), a solid dispersion of SIMVA and DL (SIMVA/DL), SIMVA-carrying CD (SIMVA-CD), or SIMVA/DL-carrying CD (SIMVA/DL-CD) in Caco-2 cells cultured for 6 hours. Each value indicates average±SD (n=6).

FIG. 3 shows that a colloidal dispersion (CD) of SIMVA including DL, P188, and Labrasol enhances cellular uptake of SIMVA in Caco-2 cells and apical sodium-dependent bile acid transporter (ASBT)-expressing or ASBT-non-expressing Madin-Darby canine kidney (MDCK) cells. (A) Confocal laser scanning microscope images of Caco-2 cells showing cellular uptake of coumarin-6 and SIMVA dispersed in water or 5% DMSO, SIMVA/DL, SIMVA-CD, and SIMVA/DL-CD. Scale bars indicate 20 μm. (B) Cellular uptake of SIMVA after 1 hour and 3 hours from treatment of Caco-2 cells with or 5% DMSO, SIMVA/DL, SIMVA-CD, or SIMVA/DL-CD. Each value indicates average±SD (n=4). Confocal laser scanning microscope images of (C) ASBT-non-expressing MDCK cells or (D) ASBT-expressing MDCK cells with regard to cellular uptake of SIMVA co-loaded with coumarin-6 in water or DMSO, SIMVA/DL, SIMVA-CD, and SIMVA/DL-CD. Scale bars indicate 20 μm. Cellular uptake of SIMVA in ASBT-expressing or ASBT-non-expressing MDCK cells after 1 hour (E) and after 3 hours (F) from treatment with SIMVA in water or SIMVA in DMSO, SIMVA/DL, SIMVA-CD, and SIMVA/DL-CD. Each value indicates average±SD (n=4). *p<0.05, **p<0.01, ***p<0.001.

FIG. 4 shows average plasma concentration-time profiles of SIMVA or platinum. (A) single treatment of SIMVA (IV or IP, 10 mg/kg) in rats; (B) SIMVA 50 mg/kg [SIMVA in water (50)], 50 mg/kg SIMVA in DMSO [SIMVA in DMSO (50)], SIMVA/DL corresponding to 50 mg/kg SIMVA [SIMVA/DL (50)], and SIMVA/DL-CD corresponding to 50 mg/kg SIMVA [SIMVA/DL-CD (50)]; (C) 10 mg/kg, 20 mg/kg, 50 mg/kg, and 100 mg/kg SIMVA [SIMVA/DL-CD (10), SIMVA/DL-CD (20), SIMVA/DL-CD (50), and SIMVA/DL-CD (100), respectively]; and (D) oral administration of 10 mg/kg oxaliplatin (OP) dispersed in water [OP (10)], ODSF containing 2.5 mg/kg, 10 mg/kg, and 20 mg/kg OP [ODSF (2.5), ODSF (10), and ODSF (20), respectively] to rats once a day for 14 days. Each value indicates average±SD (n=4).

FIG. 5 shows (A) in vitro cytotoxicity of SIMVA in CT26 cells by SIMVA in water, DL, CD, SIMVA/DL, SIMVA-CD, and SIMVA/DL-CD. Data is expressed as average±SD (n=4/group). (B) In vitro cytotoxicity to CT26 cancer cell lines of 20 UM SIMVA and SIMVA/DL-CD under treatment conditions of 0% FBS (left) or 1% FBS (right). Percentage of cell population was analyzed by annexin V-PI. Each value indicates average±SEM (n=6).

FIG. 6 shows antitumor effects of various SIMVA formulations in a CT26 tumor model. (A) Treatment schedule for administration of SIMVA formulation. (B) Average tumor growth (left) and individual tumor growth (right) under the following therapy: intraperitoneal administration (IP) of 20 mg/kg SIMVA in DMSO once-a-day [SIMVA-IP (20)]; oral administration of 20 mg/kg SIMVA in water once-a-day [SIMVA in water (20)]; oral administration of 20 mg/kg SIMVA in DMSO once-a-day [SIMVA in DMSO (20)]; oral administration of SIMVA/DL-CD once-a-day at a concentration of 10 mg/kg SIMVA [SIMVA/DL-CD (10)], 20 mg/kg SIMVA [SIMVA/DL-CD (20)], and 50 mg/kg SIMVA [SIMVA/DL-CD (50)]. Each value indicates average±SEM (n=10 in each group). ***p<0.001, **p<0.01, *p<0.05. (C) Weight of each group. (D) weight of mice treated with SIMVA/DL-CD (10) and SIMVA/DL-CD (50) in FIG. 5. (E) Ratio of CD4⁺ T cells (left) and regulatory T cells (right) in immune cells.

FIG. 7 shows in vivo antitumor effects of SIMVA formulations in CT26 tumor-bearing mice. (A) Curve of growth of CT26 tumor-bearing mice (left) and tumor weight (right). Each value indicates average±SEM (n=10 in each group). (B) Percentage of immune cells in TME. Data is expressed as average±SEM. (C) Ratio of immune cells between CD8⁺ T cells (left) and functional CD8⁺ T cells expressing CD107α⁺ (right). (D) Percentage of myeloid-derived suppressor cells (MDSC, left) and M2-like macrophages (right) among immune cells. *p<0.05, **p<0.01, ***p<0.001.

FIG. 8 shows flow cytometry analysis gating strategies to distinguish various immune cells in TMEs. Total immune cells were gated by forward scatter plot first and then side scatter plot, and were then gated to a CD45.2⁺ population. These were further gated for other subsets of interest such as CD8⁺ T cells or CD4⁺ T cells using a CD3 T cell marker. Functional CD8⁺ T cells were classified by additionally applying CD107a, and regulatory T cells were identified using CD25. For analysis of macrophage population, CD11b and F4/80 were added together, and M2 macrophages were additionally phenotyped based on expression of CD206. CD11b and Gr-1 were used to classify the myeloid-derived suppressor cells. All data was analyzed by FlowJo software.

FIG. 9 shows antitumor effects of 10 mg/kg ODSF and 10 mg/kg SIMVA/DL-CD in a CT26.CL25 tumor model. (A) Treatment schedule for administration of SIMVA/DL-CD and ODSF. (B) Curve of tumor growth in CT26.CL25-bearing mice treated with SIMVA/DL-CD and ODSF (left) and tumor weight (right). Data indicates average±SEM (n=8 to 13). (C) Change of body weight of CT26.CL25-beating mice. (D) Ratio of CD8+ T cells, regulatory T cells, and MDSC in immune cells. Data indicates average±SEM (from left to right). *p<0.05, **p<0.01, ***p<0.001.

FIG. 10 shows in vivo anticancer effects due to co-administration of ODSF and oral SIMVA/DL-CD in CT26 tumor-bearing mice. 20 mg/kg SIMVA [SIMVA/DL-CD (20)] and 10 mg/kg OXA [ODSF (10)] were orally administered alone or in combination [SIMVA/DL-CD (20)+ODSF (10)] for 21 days. (A) Tumor growth suppression of each group (left) and weight of isolated tumor (right). Each value indicates average±SEM (n=10 in each group). *p<0.05, **p<0.01, ***p<0.001. (B) Representative immunofluorescent images of CD8+ T cells in CT26 tumor tissue of tumor-bearing mice of FIG. 10A. (C)-(E) Representative mIHC microscope images of CT26 tumor tissue of tumor-bearing mice treated with SIMVA/DL-CD (20)+ODSF (10) and untreated control mice. (C) Infiltration of CD8⁺ T cells in CT26 tumor tissue. Scale bar=100 μm; (D) Expression of PD-L1 in CD11b⁺ cells and tumor tissue. Scale bar=500 μm; and (E) Enlarged image of FIG. 10D showing co-localization of CD11b⁺ cells and expression of PD-L1. Scale bars indicate 50 μm.

FIG. 11 shows antitumor effects of ODSF and SIMVA/DL-CD in CT26 tumor model of FIG. 10. (A) Weight of CT26-bearing mice during a process of administering SIMVA/DL-CD and ODSF. Data is expressed as average±SEM. (B) Growth of individual CT26 tumor during the process of administering ODSF and/or SIMVA/DL-CD. (C) Representative mIHC images of CT26 tumors of tumor-bearing mice administered with SIMVA/DL-CD (20)+ODSF (10). Scale bars indicate 800 μm and 400 μm, respectively.

FIG. 12 shows H&E staining results to identify necrosis and damage of tissue after oral administration of the control, SIMVA/DL-CD containing 20 mg/kg SIMVA [SIMVA/DL-CD (20)], ODSF containing 5 mg/kg OP [ODSF (5)], and ODSF containing 10 mg/kg OP [ODSF (10)] alone or a combination of SIMVA/DL-CD (20)+ODSF (10) and SIMVA/DL-CD (20)+ODSF (5) once a day for 21 days. Scale bars indicate 100 μm.

FIG. 13 shows effects of administration of ODSF and SIMVA/DL-CD on overcoming resistance to anti-programmed cell death protein-1 (αPD-1) in a CT26 tumor model. (A) Treatment schedule for administration of SIMVA/DL-CD (20)+ODSF (10), αPD-1, and SIMVA/DL-CD (20)+ODSF (10)+αPD-1. (B) Curve of growth of tumor growth in a CT26 mouse model co-administered with SIMVA/DL-CD (20), ODSF (10), and αPD-1. Each value indicates average±SEM (n=6 to 8 in each group). *p<0.05, **p<0.01, ***p<0.001. (C) Kaplan-Meier survival curve of CT26 mouse model co-administered with SIMVA/DL-CD (20), ODSF (10) and αPD-1 (n=6 to 8 in each group). (D) Growth of CT26 tumor formed from tumor-bearing mice and tumor-free mice of rechallenged group. Data is expressed as average±SEM (n=6 in control mice).

### BEST MODE FOR CARRYING OUT THE INVENTION

Hereinafter, the present invention will be described in detail. Meanwhile, each of the descriptions and embodiments disclosed herein may be applied to describe different descriptions and embodiments. That is, all of the combinations of various factors disclosed herein belong to the scope of the present invention. Furthermore, the scope of the present invention should not be limited by the detailed descriptions provided hereinbelow.

An aspect of the present invention to achieve the above-described objects provides a composition for preventing or treating a cancer including i) a lipophilic statin; ii) a composite of deoxycholic acid (DOCA) or a pharmaceutically acceptable salt thereof and an amino acid or an acceptable salt thereof; and iii) a surfactant, as active ingredients.

As used herein, the term “statins” refers to compounds known for anticancer effects. Specifically, statins are compounds suppressing 3-hydroxy-3-methylglutaryl-coenzyme A reductase, which regulates the first step of the mevalonate pathway involved in synthesis of cholesterol and isoprenoid lipids. The isoprenoid lipids (e.g., farnesyl or geranylgeranyl) induce prenylation of proteins, and prenylated proteins are immobilized on the inner lobules of cell membranes and initiate various signaling pathways involved in oncogenesis including mitogen-activated protein kinase, p53, and phosphoinositide 3-kinase pathways.

In the present invention, the lipophilic statin may include at least one selected from the group consisting of simvastatin (SIMVA), atorvastatin, lovastatin, fluvastatin, cerivastatin, and pitavastatin, without being limited thereto, but may specifically be simvastatin.

As used herein, “simvastatin (SIMVA)”, as a highly lipophilic lipid-lowering prodrug extracted from statins, has been known to have superior antitumor effects to hydrophilic statins not only in cohort studies but also under a variety of in vitro and in vivo experimental conditions. However, simvastatin is poorly water-soluble and undergoes various absorption processes and extensive first pass metabolism via cytochrome 3A (CYP3A) in the intestines and liver, resulting in a low in vivo oral bioavailability of approximately 5%, and thus it is difficult to apply simvastatin to clinical trials.

As used herein, “deoxycholic acid (DOCA)”, well known as a biological surfactant derived from bile acids, emulsifies fats (triglycerides), enabling a lipase (enzyme) to degrade the fat into fatty acid and glycerol. Deoxycholic acid, in which a hydrophobic part and a hydrophilic part coexist in a single molecule, acts like a surfactant to surround fat and form a micelle, exhibiting a solubilization effect. However, the solubilization effect of deoxycholic acid is low due to strong hydrophobicity and a lower molecular weight than general synthetic surfactants.

As used herein, the term “pharmaceutically acceptable salt” refers to all salts of the compound of the present invention physiologically acceptable by a target individual, and may be a formulation of the compound without causing serious irritation to an organism to which the compound is administered and impairing the biological activity and physical properties of the compound. The pharmaceutically acceptable salt may include acid addition salts of the compound formed by acids capable of forming a non-toxic acid addition salt containing a pharmaceutically acceptable anion, e.g., inorganic acids such as hydrochloric acid, sulfuric acid, nitric acid, phosphoric acid, hydrobromic acid, and hydroiodic acid; organic carbonic acids such as tartaric acid, formic acid, citric acid, acetic acid, trichloroacetic acid, trifluoroacetic acid, gluconic acid, benzoic acid, lactic acid, fumaric acid, maleic acid, and salicylic acid; or sulfonic acids such as methanesulfonic acid, ethanesulfonic acid, benzenesulfonic acid, and p-toluenesulfonic acid. For example, examples of pharmaceutically acceptable carboxylic acid salts include salts of alkali metals or alkaline earth metals such as lithium, sodium, potassium, calcium, and magnesium, salts of amino acids such as lysine, arginine, and guanidine, organic salts such as dicyclohexylamine, N-methyl-D-glucamine, tris(hydroxymethyl)methylamine, diethanolamine, choline, and triethylamine.

Also, the pharmaceutically acceptable salt of deoxycholic acid may include at least one selected from the group consisting of a hydrochloride, a sodium salt, a potassium salt, and an ammonium salt, without being limited thereto.

In the present invention, the amino acid may be a cationic amino acid, and the cationic amino acid may include at least one selected from the group consisting of L-lysine, L-arginine, and L-histidine, without being limited thereto.

In addition, as used herein, the term “acceptable” refers to the quality of being physiologically acceptable without causing an allergic reaction or similar reactions when administered to humans. The acceptable salt of an amino acid may include, for example, at least one selected from the group consisting of a hydrochloride, a sodium salt, a potassium salt, and an ammonium salt, without being limited thereto.

The deoxycholic acid or a pharmaceutically acceptable salt thereof may be chemically bonded to the amino acid or an acceptable salt thereof to form a composite. The chemical bond may be an ionic bond or a covalent bond, but is not limited thereto.

The composite of deoxycholic acid (DOCA) or a pharmaceutically acceptable salt thereof and an amino acid or an acceptable salt thereof may include at least one composite selected from the group consisting of: a composite of deoxycholic acid and the amino acid; a composite of deoxycholic acid and an acceptable salt of the amino acid; a composite of the pharmaceutically acceptable salt of deoxycholic acid and the amino acid; and a composite of the pharmaceutically acceptable salt of deoxycholic acid and the acceptable salt of the amino acid.

Since the composite consists of deoxycholic acid or a pharmaceutically acceptable salt, and an amino acid or an acceptable salt thereof, solubility of deoxycholic acid may be increased.

In view of the objects of the present invention, the composite of deoxycholic acid (DOCA) or a pharmaceutically acceptable salt thereof and an amino acid or an acceptable salt thereof may be Na-deoxycholyl-I-lysyl-methylester (DL).

In the present invention, the surfactant may be a non-ionic surfactant, for example, poloxamer, polyvinylpyrrolidone, caprylocaproyl macrogol-8 glyceride (Labrasol), alkyl polysaccharide, Cremophor, glycerol monocaprylocaprate (Capmul MCM), lauroyl macrogol-32 glyceride (Gelucire 44/14), Solutrol, polysorbate (Tween), sorbitan monolaurate (Span), propylene glycol monocaprylate (Capryol 90), propylene glycol dicaprylocaprate (Labrafac PG), oleoryl macrogol-6 glyceride (Labrafil M1944 CS), lauroyl macrogol-6 glyceride (Labrafil M2130 CS), linoleoyl macrogol-6 glyceride (Labrafil M2125 CS), medium-chain triglyceride (Labrafac), oleic acid, stearic acid, glyceryl behenate (Compritol 888), glycerol monostearate, polyethylene glycol, ethoxylate, alkanoamide, amine oxide, acetylene glycol, sugar ester, sorbitan ester, glycerol ester, or glycoside, specifically, poloxamer or caprylocaproyl macrogol-8 glyceride, without being limited thereto.

The lipophilic statin, deoxycholic acid or the pharmaceutically acceptable salt thereof, the amino acid or the acceptable salt thereof, and the surfactant may be commercially purchased or manufactured by way of a chemical synthesis method well known in the art.

In the present invention, i) the lipophilic statin; ii) the composite of deoxycholic acid (DOCA) or a pharmaceutically acceptable salt thereof and an amino acid or an acceptable salt thereof; and iii) the surfactant may constitute a micelle shape.

In a specific embodiment of the present invention, in order to improve solubility and intestinal membrane permeability of simvastatin (SIMVA), a colloidal dispersion (CD) was prepared by using the composite of deoxycholic acid (DOCA) or a pharmaceutically acceptable salt thereof and an amino acid or an acceptable salt thereof, i.e., Na-deoxycholyl-I-lysyl-methylester (DL), as an oral permeation enhancer, and caprylocaproyl macrogol-8 glyceride (Labrasol) and poloxamer 188 (P188), as surfactants for improving dispersibility and inhibiting coagulation. Then, the colloidal dispersion was lyophilized to completely remove water, thereby preparing SIMVA/DL-CD.

A weight ratio of SIMVA:DL constituting SIMVA/DL-CD may be from 1:1.00 to 1:3.00, specifically from 1:1.20 to 1:2.90, more specifically from 1:1.40 to 1:2.88, even more specifically 1:2.88. A weight ratio of SIMVA:DL:Labrasol:P188 constituting SIMVA/DL-CD may be 1:2.88:1.5:2.

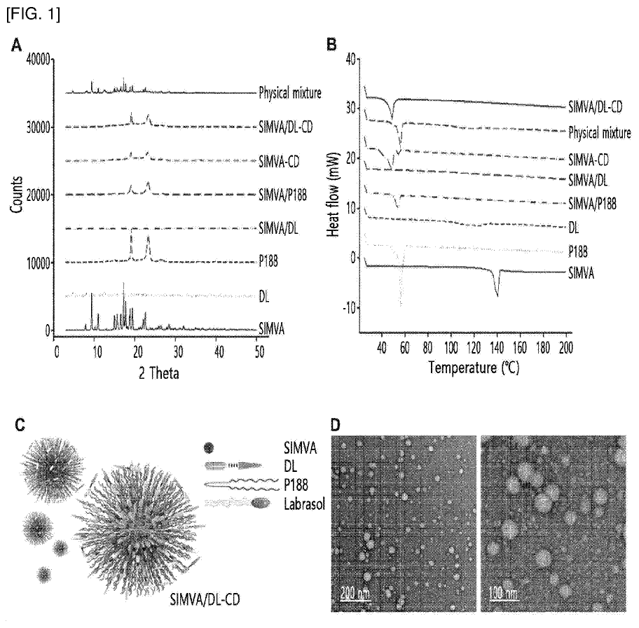

In addition, based on analysis results of particle size, polydispersity (PDI), and zeta potential, it was confirmed that SIMVA was successfully included in SIMVA/DL-CD (Table 2) and it was confirmed that SIMVA of SIMVA/DL-CD was present in an amorphous state (FIGS. 1A and 1B).

In transmission electron microscope (TEM) images of SIMVA/DL-CD, formation of spherical objects with a uniform diameter less than 100 nm is observed (FIG. 1D), and SIMVA/DL-CD has a particle size of 125 nm+1.78 nm and a PDI of 0.13±0.02, which are lower than SIMVA/Labrasol in which SIMVA and Labrasol are dispersed, SIMVA/P188 in which SIMVA and P188 are dispersed, and SIMVA/DL in which SIMVA and DL are dispersed by 1.40 times, 12.8 times, and 16.5 times, respectively (Table 2).

The composition of the present invention may have significantly improved solubility and permeability of the lipophilic statin.

In a specific embodiment of the present invention, SIMVA/DL-CD has improved solubility and effective permeability (Pₑ) of SIMVA compared with those of SIMVA/DL by 418% and 4.58 times, respectively. In addition, the size of SIMVA/DL-CD is less than that of SIMVA/DL by 15.8 times. The size reduction to the nanoparticle range may be caused by linkage to Labrasol and P188, and absorption of SIMVA may further be improved thereby. In addition to the improved solubility and permeability, it was found from analysis that positively charged SIMVA/DL-CD permeate more into negatively charged artificial phospholipid membranes by interaction between charges.

Solubility and permeability of SIMVA/DL-CD were improved compared to SIMVA/DL/Labrasol by 208% and 404%, respectively. Thus, improved solubility due to the amorphous property of SIMVA/DL-CD did not reduce free fraction of a drug. Further, permeability remained unaffected and exhibited a solubility-dependent increment even through the monolayers.

In addition, it was confirmed that in SIMVA/DL/Labrasol and SIMVA/DL-CD, a structural orientation of DL increases interaction with apical sodium-dependent bile acid transporter (ASBT) and promotes infiltration through cell membranes by ASBT-mediated endocytosis.

The composition of the present invention may have significantly improved in vivo oral bioavailability of the lipophilic statin.

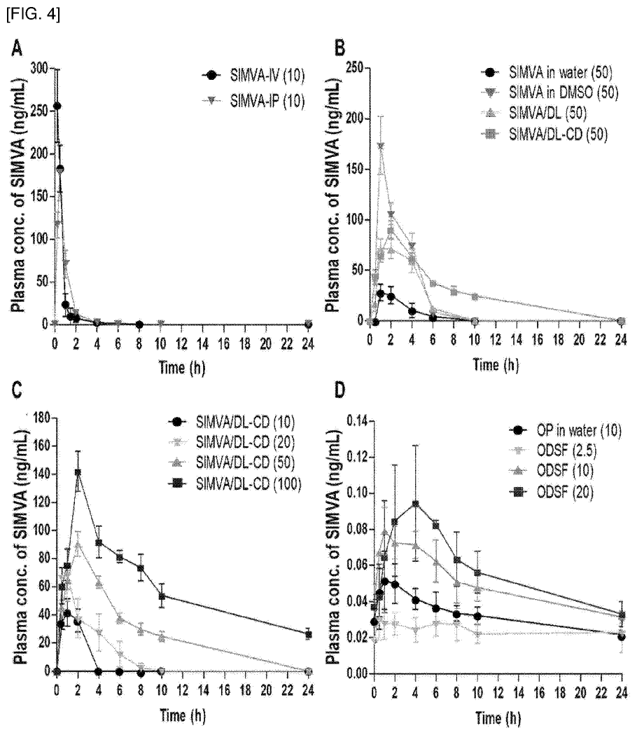

In a specific embodiment of the present invention, after oral administration of 50 mg/kg SIMVA [SIMVA in DMSO (50)], a maximum plasma concentration (Cₘₐₓ) was improved by 6.21 times, and an area under a plasma concentration-time curve (0-24 hours) (AUCₗₐₛₜ) increased by 558% compared to those of SIMVA in water (50). The in vivo oral bioavailability was improved by 863% compared to that of SIMVA in water (50) (FIG. 4B and Table 4). The improvement of in vivo oral bioavailability of SIMVA in DMSO (50) was found from analysis to cause improved solubility and a high absorption property of DMSO in the intestinal epithelium.

SIMVA/DL-CD (50) for oral administration including 50 mg/kg SIMVA exhibited superior in vivo oral bioavailability to SIMVA/DL (50) by 168%, indicating synergistic effects of P188 and Labrasol on in vivo oral bioavailability.

The pharmaceutical composition of the present invention has a use for “prevention” and/or “treatment” of a cancer. For preventive use, the pharmaceutical composition of the present invention may be administered to an individual having or suspected to have a risk of developing a disease, disorder, or condition described herein. For therapeutic use, the pharmaceutical composition of the present invention may be administered to an individual such as a patient already suffering from the disease, disorder, or condition described herein in an amount sufficient to treat or at least partially cease symptoms of the disease, disorder, or condition. An effective amount for this use may vary according to severity and progression of the disease, disorder, or condition, previous history of treatment, health status and drug sensitivity of an individual, and judgement of doctors or veterinarians.

In the present invention, the cancer may be any cancer known in the art without limitation, for example, lung cancer (e.g., non-small cell lung cancer, small cell lung cancer, and malignant mesothelioma), mesothelioma, pancreatic cancer (e.g., pancreatic ductal cancer and pancreatic endocrine tumor), pharyngeal cancer, laryngeal cancer, esophagus cancer, gastric cancer (e.g., papillary adenocarcinoma, mucinous adenocarcinoma, and adenosquamous carcinoma), duodenal cancer, small intestinal cancer, colorectal cancer (e.g., colon cancer, rectal cancer, anal cancer, hereditary colorectal cancer, hereditary non-polypasis colorectal cancer, and gastrointestinal stromal tumor), breast cancer (e.g., invasive ductal carcinoma, non-invasive ductal carcinoma, and inflammatory breast cancer), ovarian cancer (e.g., ovarian epithelial carcinoma, extragonadal germ cell tumor, ovarian germ cell tumor, and ovarian low malignant potential tumor), testis tumor, prostate cancer (e.g., hormone-dependent prostate cancer and hormone-independent prostate cancer), liver cancer (e.g., hepatoma, primary liver cancer, and extrahepatic bile duct cancer), thyroid cancer (e.g., medullary thyroid carcinoma), renal cancer (e.g., renal cell carcinoma and transitional cell carcinoma in kidney and ureter), uterine cancer (e.g., cervix cancer, corpus uteri cancer, and uterus sarcoma), brain tumor (e.g., medulloblastoma, glioma, pineal astrocytoma, pilocytic astrocytoma, diffuse astrocytoma, anaplastic astrocytoma, and hypophyseal adenoma), retina blastoma, skin cancer (e.g., basalioma and malignant melanoma), sarcoma (e.g., rhabdomyosarcoma, leiomyosarcoma, and soft tissue sarcoma), malignant bone tumor, urinary bladder cancer, hematologic cancer (e.g., multiple myeloma, leukemia, malignant lymphoma, Hodgkin's disease, and chronic myeloproliferative disease), cancer of unknown primary, or the like.

The pharmaceutical composition of the present invention may further include an appropriate carrier, excipient, or diluent commonly used in preparation of the pharmaceutical composition. In this regard, an amount of the lipophilic statin, as an active ingredient contained in the pharmaceutical composition, is not particularly limited, but may be in the range of 0.1 wt % to 90 wt %, specifically 1 wt % to 50 wt %, based on a total weight of the composition.

The pharmaceutical composition may be formulated into any formulation for oral or parenteral administration selected from the group consisting of tablets, pills, powders, granules, capsules, formulations for internal use, syrups, sterile aqueous solutions, non-aqueous solvents, suspensions, emulsions, lyophilizates, and suppositories. For formulations, a diluent or excipient commonly used in the art such as a filler, an extender, a binder, a humectant, a disintegrant, and a surfactant may be used. Solid formulations for oral administration may include tablets, pills, powders, granules, and capsules, and the formulations for oral administration may further include a pharmaceutical additive such as a diluent, a binder, a swelling agent, and a lubricant. In addition to simple excipients, a lubricant such as magnesium stearate and talc are also used.

For example, the diluent includes, but is not limited to, lactose, dextrin, mannitol, sorbitol, starch, microcrystalline cellulose, calcium hydrogen phosphate, anhydrous calcium hydrogen phosphate, calcium carbonate, saccharide, and the like.

For example, the binder includes, but is not limited to, polyvinylpyrrolidone, copovidone, gelatin, starch, sucrose, methyl cellulose, ethyl cellulose, hydroxyethyl cellulose, hydroxypropyl cellulose, hydroxypropylalkyl cellulose, and the like.

The swelling agent includes at least one selected from the group consisting of crosslinked polyvinylpyrrolidone, crosslinked sodium carboxymethyl cellulose, crosslinked calcium carboxymethyl cellulose, crosslinked carboxymethyl cellulose, sodium starch glycolate, carboxymethyl starch, sodium carboxymethyl starch, a potassium methacrylate-divinylbenzene copolymer, amylose, crosslinked amylose, starch derivative, microcrystalline cellulose and cellulose derivatives, and cyclodextrin and dextrin derivatives.

For example, the lubricant includes, but is not limited to, stearic acid, stearate, talc, corn starch, carnauba wax, light anhydrous silicic acid, magnesium silicate, synthetic aluminum silicate, hardened oil, white beeswax, titanium oxide, microcrystalline cellulose, Macrogol 4000 and 6000, myristic acid isopropyl, calcium hydrogen phosphate, talc, and the like.

Liquid formulations for oral administration may be formulations for internal use, syrups, and the like, and may include various excipients, such as a humectant, a sweetener, an aromatic, and a preservative in addition to a conventional diluent such as water or liquid paraffin.

Formulations for parenteral administration may include a sterile aqueous solution, a non-aqueous solvent, a suspension, an emulsion, a lyophilizate, and a suppository. The non-aqueous solvent and the suspension may include propylene glycol, polyethylene glycol, vegetable oil such as olive oil, and injectable ester such as ethyloleate. The suppository may be Witepsol, Macrogol, Tween 61, cacao butter, laurin butter, glycerogelatin, or the like. The injections may be prepared by using an aqueous solvent such as a physiological saline solution and Ringer's solution, or a non-aqueous solvent such as vegetable oil, a higher fatty acid ester (e.g., ethyl oleate), and alcohol (e.g., ethanol, benzyl alcohol, propylene glycol, and glycerin), and may include a pharmaceutical carrier such as a stabilizer for preventing degradation (e.g., ascorbic acid, sodium bisulfite, sodium pyrosulfite, BHA, tocopherol, and EDTA), an emulsifier, a buffer for adjusting the pH, and a preservative for inhibiting microbial growth (e.g., phenylmercury nitrate, thimerosal, benzalkonium chloride, phenol, cresol, and benzyl alcohol).

The pharmaceutical composition of the present invention may be administered in a pharmaceutically effective amount.

As used herein, the term “pharmaceutically effective amount” refers to an amount sufficient to treat a disease at a reasonable benefit/risk ratio applicable to any medical treatment. An effective dosage level may be determined according to factors including type of individual, severity of illness, age and gender of individual, type of disease, activity of a drug, sensitivity to a drug, administration time, administration route, and excretion rate, treatment duration, a drug to be mixed with or concurrently used in combination with the composition of the present invention, and other factors well known in the medical field. The composition of the present invention may be administered alone or in combination with other therapeutic agents sequentially or simultaneously with existing therapeutic agents. The composition may be administered in a single- or multiple-dosage form. It is important to administer the composition in a minimum amount capable of exhibiting a maximum effect without causing side effects, considering all of the above-described factors. The amount may be readily determined by those skilled in the art.

Specifically, the pharmaceutical composition of the present invention may be co-administered with an anticancer drug or used in combination with an anticancer therapy. The anticancer drug may be, for example, an alkylating agent, a platinum-based drug, an antimetabolite, an antibiotic, a Taxane-based anticancer drug, a Vinca alkaloid-based anticancer drug, a targeted agent, an immune anticancer drug, a cancer vaccine, a cell therapy product, an oncolytic virus, and any combination thereof, and the anticancer therapy may be radiotherapy, photodynamic therapy, or the like. However, the present invention is not limited thereto and may include any anticancer drug or anticancer therapy well known in the art.

Examples of the alkylating agent may include nitrogen mustard-based drugs including Mechlorethamine (nitrogen mustard), Cyclophosphamide, Ifosfamide, Melphalan, and Chlorambucil; ethyleneimine- and methylmelamine-based drugs including Thiotepa and Altretamine; methylhydrazine derivatives including Procarbazine; alkyl sulfonate-based drugs including Busulfan; nitrosourea-based drugs including Carmustine (BCNU) and Lomustine (CCNU); and triazine-based drugs including Dacarbazine (DTIC), without being limited thereto.

Examples of the platinum-based drug may include Cisplatin, Carboplatin, and Oxaliplatin, without being limited thereto.

Examples of the antimetabolite may include folate antagonist-based drugs, purine antagonist-based drugs, and pyrimidine antagonist-based drugs, without being limited thereto.

Examples of the antibiotic may include Etoposide, Topotecan, Irinotecan, Idarubicin, Epirubicin, Dactinomycin, Doxorubicin, Adriamycin, Daunorubicin, Bleomycin, Mitomycin C, and Mitoxantrone, without being limited thereto.

Examples of the Taxane-based anticancer drug may include Paclitaxel and Docetaxel, without being limited thereto.

Examples of the Vinca-based anticancer drug may include Vincristine, Vinblastine, and Vinorelbine, without being limited thereto.

Examples of the targeted agent may include an epidermal growth factor receptor (EGFR) targeted agent, a human epidermal growth factor receptor 2 (HER2) targeted agent, a B cell marker (CD20) targeted agent, a myeloid cell surface antigen (CD33) targeted agent, a cluster of differentiation 52 (CD52) targeted agent, a tumor necrosis factor receptor superfamily member 8 (CD30) targeted agent, a breakpoint cluster region protein-tyrosine-protein kinase (bcr-abl)/tyrosine kinase receptor (c-Kit) targeted agent, an anaplastic lymphoma receptor tyrosine kinase (ALK) targeted agent, an antiangiogenic targeted agent, a mammalian target of rapamycin (mTOR) targeted agent, a cyclin-dependent kinase 4/6 (CDK4/6) targeted agent, a poly (ADP-ribose) polymerase (PARP) targeted agent, a proteasome inhibitor, a tyrosine kinase antagonist, a protein kinase C inhibitor, and a farnesyl transferase inhibitor, without being limited thereto.

Examples of the immune anticancer drug may include an anti-programmed cell death protein-1 (PD-1) interaction inhibitor, an anti-programmed cell death ligand-1 (PD-L1) interaction inhibitor, a cytotoxic T lymphocyte associated antigen 4 (CTLA4 or CD152)/B7-1/B7-2 interaction inhibitor, and a cluster of differentiation 47 (CD47)/signal-regulatory protein (SIRP) interaction inhibitor, without being limited thereto. The interaction inhibitor may be, for example, an antibody.

Although a preferred dosage of the composition of the present invention varies according to condition and body weight of a patient, severity of disease, formulation of a drug, administration route, and administration period, the composition of the present invention may be administered such that the amount of the lipophilic statin is in the range of 0.01 mg/kg to 100 mg/kg, specifically 0.1 mg/kg to 50 mg/kg per day for desired effects. Administration may be performed once a day or several times a day in divided doses. The composition may be administered to mammals such as rats, livestock, and humans via various routes. The composition of the present invention may be administered via any general route as long as the composition reaches a target tissue. For example, the composition may be administered via oral administration, intraperitoneal administration, intravenous administration, intramuscular administration, subcutaneous administration, intradermal administration, topical administration, intranasal administration, intrapulmonary administration, and rectal administration, specifically oral administration, without being limited thereto. In addition, the pharmaceutical composition of the present invention may be administered by any device capable of delivering the active ingredient to a target cell.

In addition, the pharmaceutical composition of the present invention may be used in the form of veterinary drugs as well as pharmaceuticals applied to humans. In this regard, animals include livestock and pets.

The composition of the present invention may exhibit cytotoxicity to cancer cells.

In a specific embodiment of the present invention, as a result of measuring IC₅₀ defined as a concentration of a drug required to kill 50% of cells during a predetermined period, SIMVA dispersed in water had an IC₅₀ of 183 μg/ml±1.52 μg/mL, SIMVA/DL had an IC₅₀ of 28.3 μg/mL+0.857 μg/mL, SIMVA-CD had an IC₅₀ of 102 μg/mL±1.11 μg/mL, and SIMVA/DL-CD had an IC₅₀ of 3.47 μg/mL±0.299 μg/mL, which is lower than those of SIMVA-CD and SIMVA/DL by 290% and 83%, respectively.

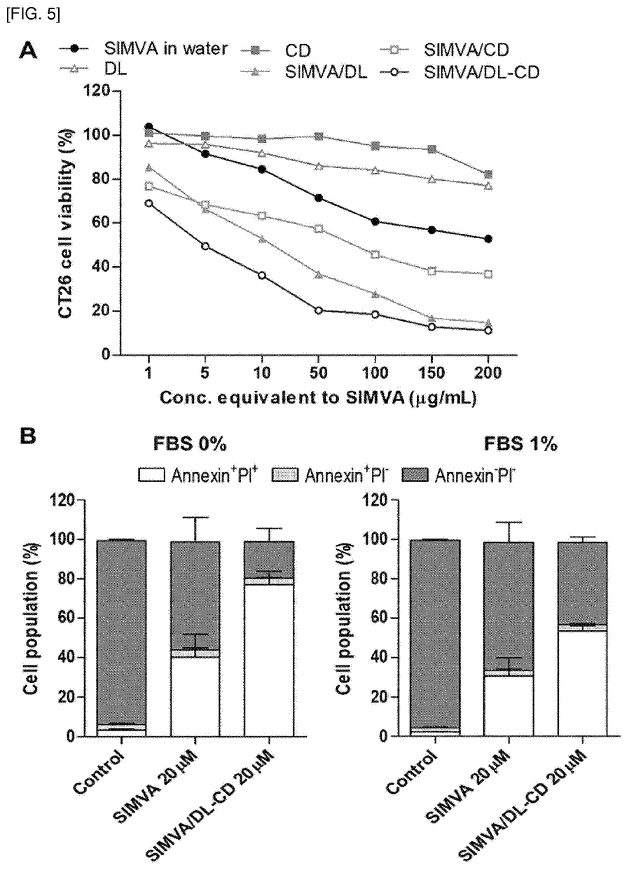

In addition, in Annexin V-Propidium iodide (PI) analysis, SIMVA/DL-CD exhibited superior cytotoxicity effects compared to SIMVA under the conditions of 0% FBS and 1% FBS (FIG. 5B).

Accordingly, it was confirmed that cancer cell viability may be significantly reduced even in the presence of serum by improving solubility and permeability of the lipophilic statin.

The composition of the present invention may have antitumor effects.

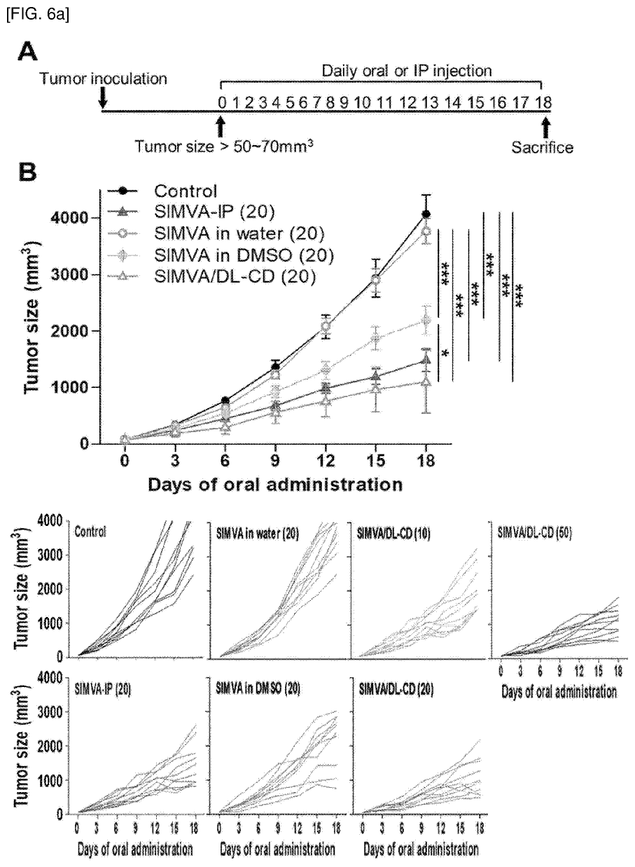

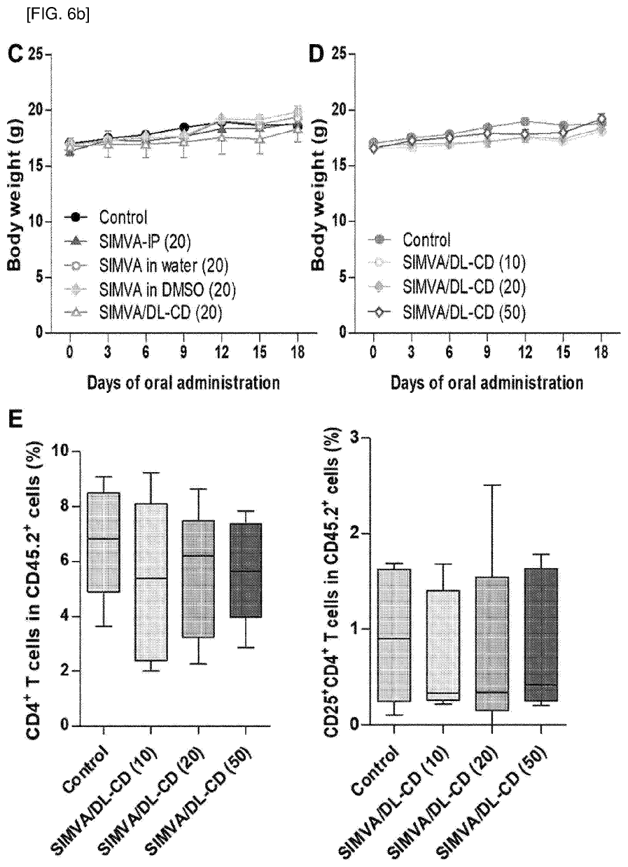

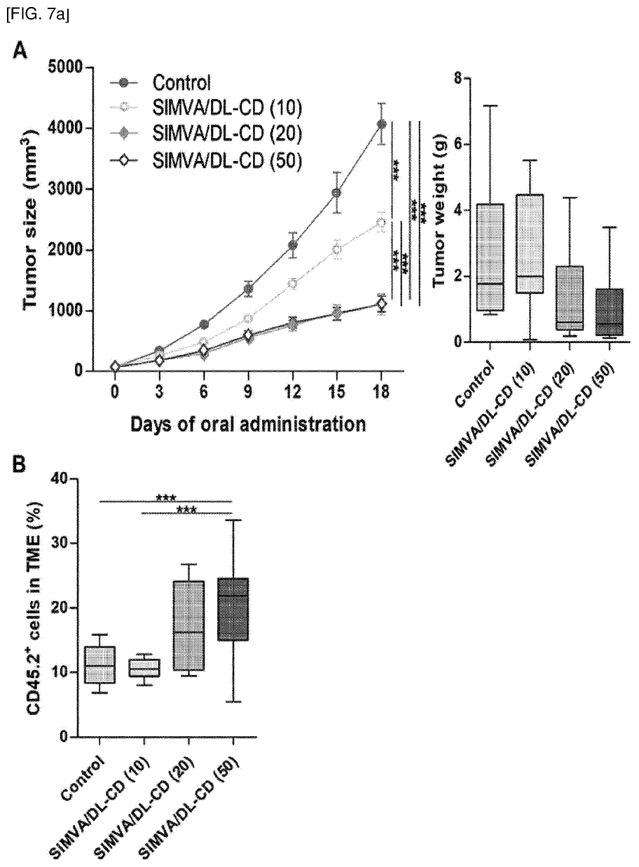

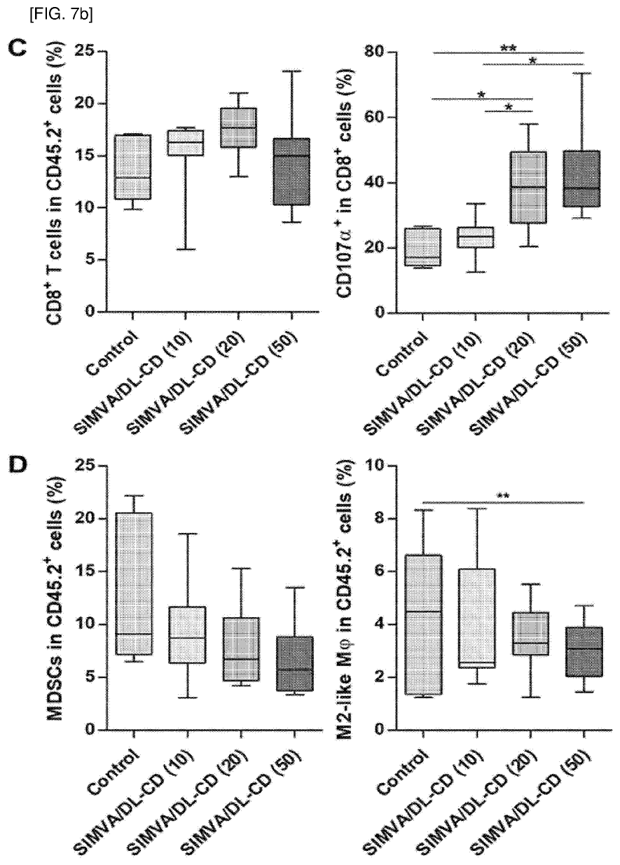

In a specific embodiment of the present invention, as a result of identifying antitumor effects in a CT26 tumor-bearing mouse model, SIMVA/DL-CD (containing 10 mg/kg SIMVA, hereinafter referred to as 10) delayed tumor growth and decreased weight of isolated tumors more than a control (untreated) or SIMVA in water (containing 20 mg/kg SIMVA), which have no anticancer effects, by 154% and 145%, respectively (FIGS. 6B and 7A). In addition, SIMVA/DL-CD (10) exhibited a tumor growth inhibition rate higher than that of SIMVA in DMSO (containing 20 mg/kg SIMVA, hereinafter referred to as 20) by 1.27 times. Based thereon, it was confirmed that improved solubility, permeability, and in vivo oral bioavailability are essential for antitumor effects of the statin.

SIMVA/DL-CD (20) delayed tumor growth more than SIMVA in DMSO (20) by 1.92 times and more than SIMVA-IP (20) by 2.46 times and lowered tumor weight more than SIMVA-IP (20) and SIMVA in DMSO (20) by 361% and 272%, respectively, without toxicity (FIGS. 6B and 6C).

These results indicate that the lipophilic statin/DL-CD may effectively deliver the lipophilic statin via an oral administration route and exhibit better antitumor effects than other solubilizers or other administration routes.

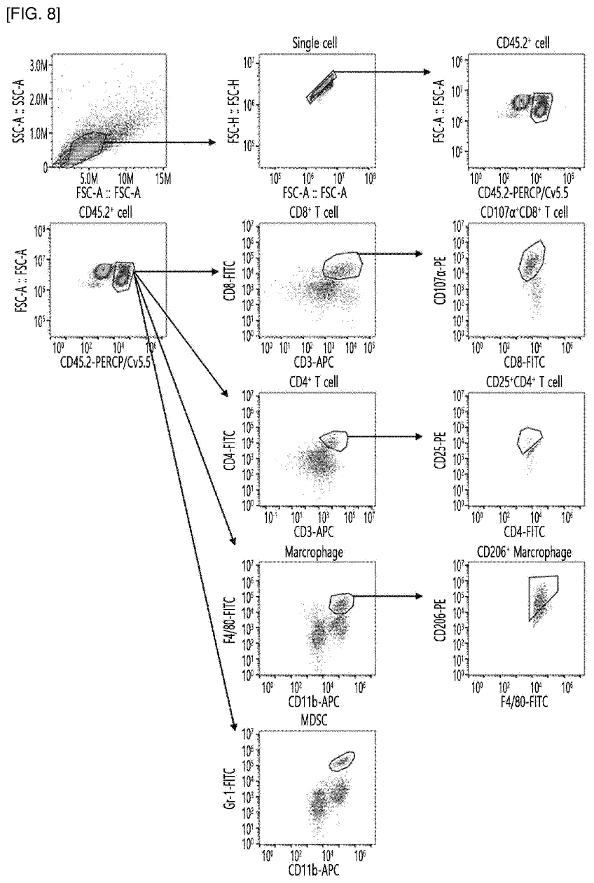

In another embodiment of the present invention, oral administration of SIMVA/DL-CD increased the percentage of immune cells in TMEs in a CT26 tumor-bearing mouse model (FIGS. 7B and 8). Although increases in CD8⁺ T cells, among the immune cell types, were significant at boundaries as the dose increased, the percentage of CD8⁺ T cells expressing cytotoxic CD107α remarkably increased due to administration of SIMVA/DL-CD (20) and SIMVA/DL-CD (containing 50 mg/kg SIMVA, hereinafter referred to as 50) (FIG. 7C).

In addition, myeloid-derived suppressor cells (MDSCs) and M2-like macrophages, serving as immunosuppressive cells, tended to decrease as a dose of SIMVA/DL-CD increased (FIG. 7D).

The results indicate that the lipophilic statin/DL-CD induces antitumor immune responses.

The composition of the present invention may improve antitumor effects when co-administered with the platinum-based anticancer drug.

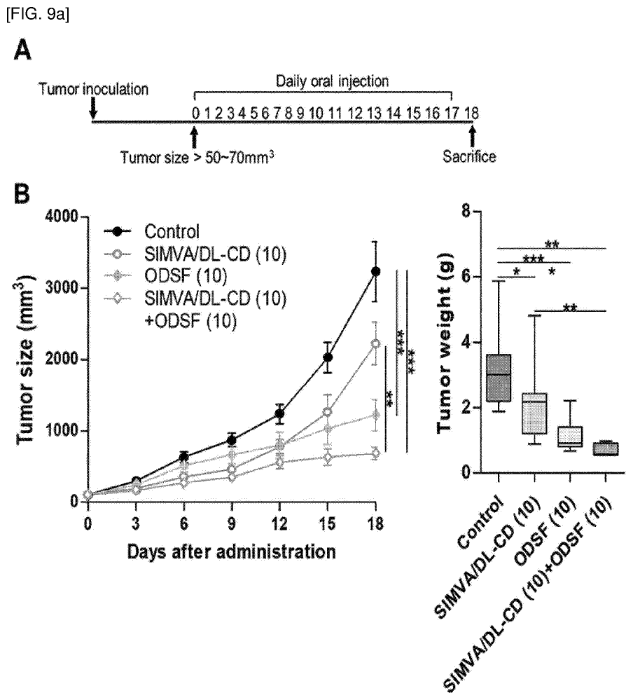

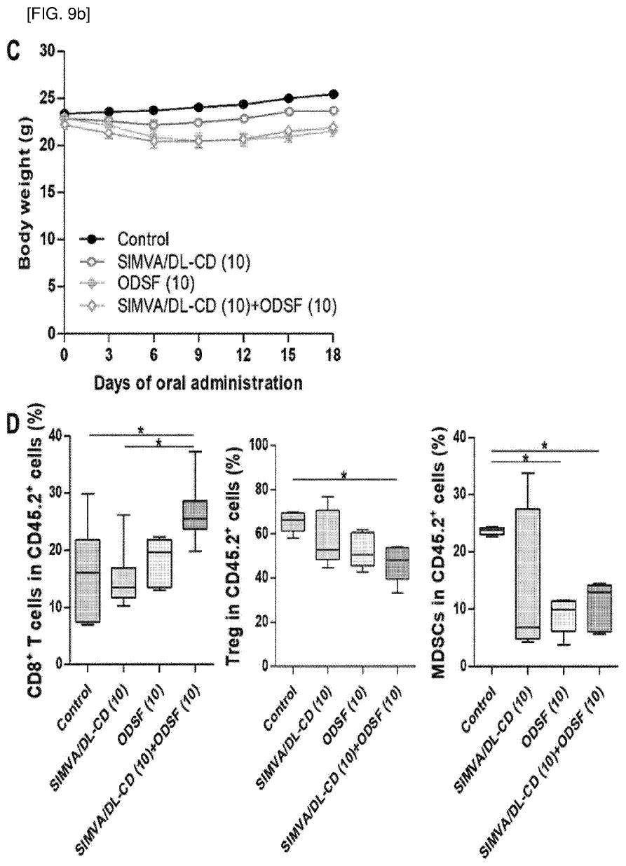

In an embodiment of the present invention, in CT26.CL25 tumors expressing immunogenic neoantigen β-galactosidase, co-administration of SIMVA/DL-CD (10) and ODSF, which is an ionic composite of oxaliplatin (OP) and DL (containing 10 mg/kg OP, hereinafter referred to as 10), exhibited superior antitumor effects compared to administration of either SIMVA/DL-CD (10) or ODSF (10) alone (FIG. 9B), no significant change in body weight was observed in mice treated therewith (FIG. 9C). The co-administration increased CD8⁺ T cell population and reduced immunosuppressive T_{reg} and MDSC in the TME (FIG. 9D).

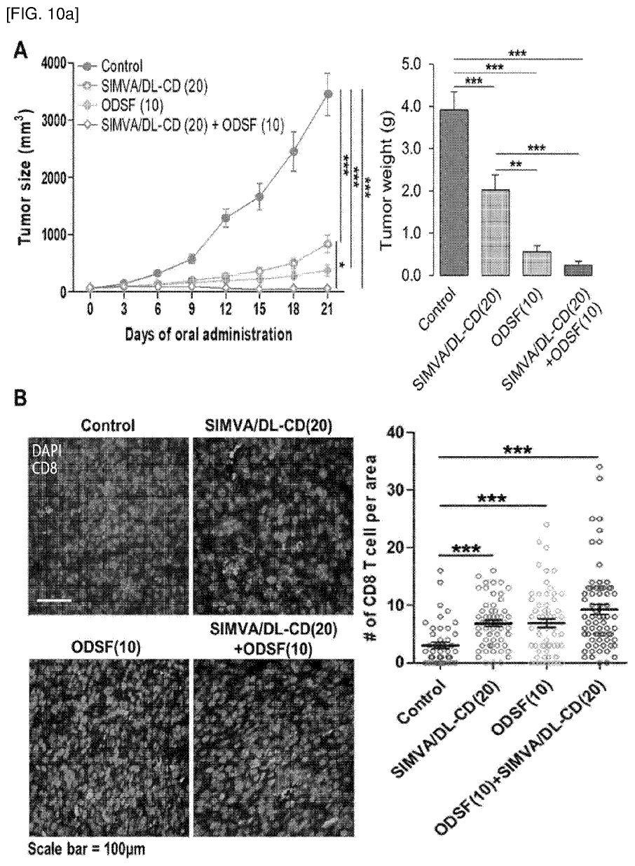

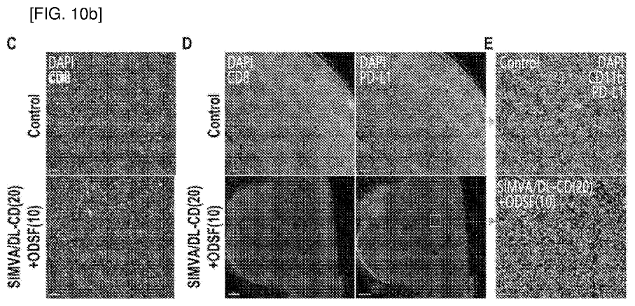

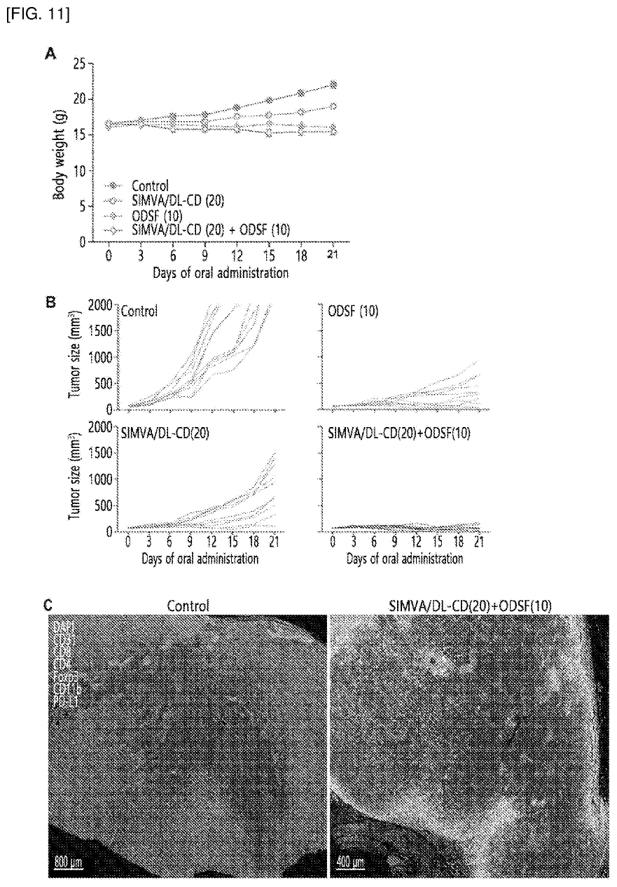

In another embodiment of the present invention, as a result of identifying in vivo antitumor effects of in vivo antitumor effect of SIMVA/DL-CD (20) alone or in combination with ODSF as 10 mg/kg OP [ODFS (10)] was investigated in CT26 cell-bearing mice through daily oral administration for three consecutive weeks, SIMVA/DL-CD (20) alone induced a 4.09-fold greater decrease in tumor size compared to the control (untreated). Inhibitory effects of the combination of SIMVA/DL-CD (20) and ODSF (10) on tumor growth in the mice were improved by 15.1 times compared with those of SIMVA/DL-CD (20) alone (FIGS. 10A, 11A, and 11B). Also, in the group treated with SIMVA/DL-CD (20)+ODSF (10), the weight of isolated tumors was lower than that observed after treatment with SIMVA/DL-CD (20) by 825%.

These results clearly indicate superior therapeutic effects of combination therapy of lipophilic statin/DL-CD and ODSF on colorectal cancer to single therapy of each formulation.

In another embodiment of the present invention, in order to identify a change in immune cells associated with antitumor effects of combination of SIMVA/DL-CD (20)+ODSF (10), tumor-infiltrating CD8⁺ T cells were subjected to immunohistochemistry on day 21 from treatment. As expected, the combination therapy induced more populations of CD8⁺ T cells than single therapy of each formulation alone, thereby increasing the number of CD8⁺ T cells in TME (FIG. 10B). As a result of additional analysis via multiplex immunohistochemistry (mIHC), the co-administration increased a density of tumor-infiltrating CD8⁺ T cells compared to the control (FIG. 10C). In addition, the co-administration limited formation of CD11b⁺ cell populations at boundaries of tumors when compared to the control (FIG. 10D). The CD11b⁺ cells were localized with expression of programmed cell death ligand (PD-L1) (FIG. 10E), indicating formation of immunosuppressing TME capable of impairing an antitumor immune response. In contrast, CD4⁺ T cells and Foxp3⁺CD4⁺ T cells were hardly detected in both TMEs.

Based on these results, it was confirmed that combination therapy of SIMVA/DL-CD and ODSF may induce strong antitumor effects by activating CD8⁺ T cell immunity against cancer, which may also be limited by PD-L1 that expresses immunosuppressive CD11b⁺ cell populations in the TME.

The composition of the present invention may have improved antitumor effects when co-administered with the platinum-based anticancer drug and the targeted agent.

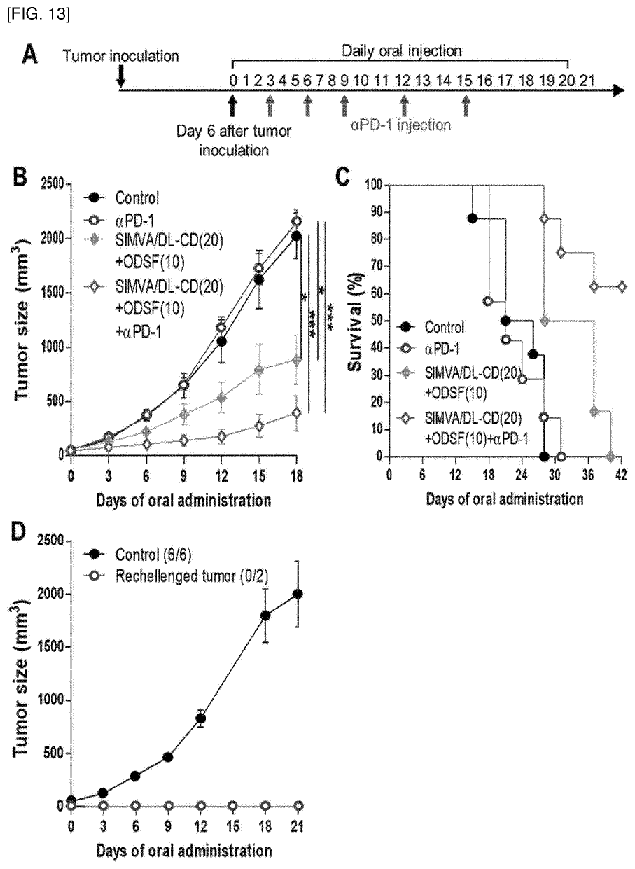

Considering that a high expression level of the programmed death-ligand-1 (PD-L1) weakens CD8⁺ T cell immunity, it was expected that a blockade of the PD-1 may improve therapeutic effects of combination therapy of SIMVA/DL-CD and ODSF. Thus, in an embodiment of the present invention, as a result of identifying antitumor effects of triple combination therapy of SIMVA/DL-CD, ODSF, and αPD-1 in a CT26 tumor-bearing mouse model known to be resistant to an immune checkpoint blockade (ICB), it was confirmed that antitumor effects were induced (FIG. 13B), and the survival of the mice was prolonged (FIG. 13C).

Based on these results, it was confirmed that the combination therapy of lipophilic statin/DL-CD and ODSF not only sensitizes ICB-resistant tumors but also acts complementarily to αPD-1 therapy to induce a continuous antitumor immune response.

Another aspect of the present invention provides a method of preparing a composition including: i) a lipophilic statin; ii) a composite of deoxycholic acid (DOCA) or a pharmaceutically acceptable salt thereof and an amino acid or an acceptable salt thereof; and iii) a surfactant.

Terms used herein are as described above.

Specifically, the method includes:
a) adding a lipophilic statin to an aqueous solution including a first surfactant, followed by stirring to prepare a dispersion;
b) adding an aqueous solution including the composite of deoxycholic acid (DOCA) or a pharmaceutically acceptable salt thereof and an amino acid or an acceptable salt thereof to the dispersion, followed by stirring to prepare a mixture; and
c) adding an aqueous solution including a second surfactant to the mixture.

The step a) may be a process of adding a lipophilic statin to an aqueous solution including a first surfactant, followed by stirring to prepare a dispersion.

The first surfactant may be a non-ionic surfactant, which is as described above, and may specifically be a poloxamer.

In an embodiment of the present invention, the poloxamer is a copolymer of polyethylene oxide and polypropylene oxide, and simvastatin was added to a P188 aqueous solution, prepared by dissolving poloxamer 188 (P188) in deionized water (DW) while continuously stirring.

The step b) may be a process of adding an aqueous solution including the composite of deoxycholic acid (DOCA) or a pharmaceutically acceptable salt thereof and an amino acid or an acceptable salt thereof to the dispersion prepared in the step a) followed by stirring to prepare a mixture.

In an embodiment of the present invention, DL was dissolved in DW, and the solution was added dropwise to the dispersion of SIMVA and P188 prepared in the step a).

The step c) may be a process of adding an aqueous solution including a second surfactant to the mixture.

The second surfactant may be a non-ionic surfactant, which is as described above, and may specifically be caprylocaproyl macrogol-8 glyceride.

In an embodiment of the present invention, Labrasol, as caprylocaproyl macrogol-8 glyceride, was added dropwise to the mixture of lipophilic statin, DL, and P188 prepared in the step b) by regular vortex mixing.

The method of the present invention may further include dehydrating after the step c).

In an embodiment of the present invention, the dehydrating was performed by freeze-drying after the step c).

The composition including i) a lipophilic statin; ii) a composite of deoxycholic acid (DOCA) or a pharmaceutically acceptable salt thereof and an amino acid or an acceptable salt thereof; and iii) a surfactant, prepared according to the method of the present invention has improved solubility and permeability of the lipophilic statin, resulting in improvement of in vivo oral bioavailability, and thus anticancer effects may be enhanced, as described above.

Another aspect of the present invention provides a method of preventing or treating a cancer, the method including administering the composition of the present invention to an individual.

Terms used herein are as described above.

As used herein, the term “individual” is not particularly limited as long as the individual has a cancer or a risk of developing a cancer or needs lipolysis. For example, animals such as human, monkeys, dogs, cats, rabbits, guinea pigs, rats, mice, cows, sheep, pigs, goats, birds, and fish may be included. Cancer may be effectively prevented or treated by administering the pharmaceutical composition of the present invention to an individual suspected to have a cancer or an individual requiring lipolysis.

As used herein, the term “administration” refers to introduction of substances for a therapy by way of any appropriate method, and the substances may be administered via various routes such as oral or parenteral routes as long as the substances are able to reach a target tissue. The administration is as described above.

The composition including i) a lipophilic statin; ii) a composite of deoxycholic acid (DOCA) or a pharmaceutically acceptable salt thereof and an amino acid or an acceptable salt thereof; and iii) a surfactant according to the present invention has improved solubility and permeability of the lipophilic statin, resulting in improvement of in vivo oral bioavailability, and thus anticancer effects may be enhanced, as described above.

Another aspect of the present invention is to provides a use of a composition for preventing or treating a cancer, comprising: i) a lipophilic statin; ii) a composite of deoxycholic acid (DOCA) or a pharmaceutically acceptable salt thereof and an amino acid or an acceptable salt thereof; and iii) a surfactant.

### MODE FOR THE INVENTION

Hereinafter, the present invention will be described in more detail with reference to the following examples. However, the following examples are merely presented to exemplify the present invention, and the scope of the present invention is not limited thereto. These examples are provided to fully convey the concept of the invention to those skilled in the art.

### Example 1: Preparation of SIMVA/DL-CD

As an oral permeation enhancer, N^{α}-deoxycholyl-I-lysyl-methylester (DL) was synthesized via conjugation of positively charged L-lysine (Lys) and deoxycholic acid (DA) according to a method disclosed in a previous document (R. Pangeni, et al., International journal of nanomedicine 2020, 15, 7719.).

Specifically, N-Boc-L-lysine methyl ester hydrochloride [H-Lys (Boc)-OMe·HCl] in N-methylmorpholine (2.7%, w/v) was added to DA (26 g) in a mixed solvent of ethyl chloroformate (6.4 mL), N-methylmorphine (7.4 mL), and tetrahydrofuran (800 mL). The mixture was stirred at room temperature overnight, and a precipitate was filtered, followed by drying by evaporation. Lys (Boc) DA was purified by column chromatography using a mixture of chloroform and methanol. The purified Lys (Boc) DA was dissolved in a mixture of acetyl chloride and methanol in an ice bath. After completely removing the organic solvent, a residue was dissolved in deionized water (DW) and washed with chloroform. An aqueous layer was collected and freeze-dried to obtain DL powder.

Subsequently, simvastatin (SIMVA) was dispersed in a DL solution (SIMVA/DL) to improve solubility and intestinal membrane permeability.

Specifically, 28.8 mg of DL dissolved in 10 mL of DW was added dropwise to 10 ml of a 1 mg/mL waterborne dispersion of SIMVA by continuous vortex mixing, followed by sonication for 15 minutes to prepare SIMVA/DL.

Subsequently, in order to further increase dispersibility of SIMVA/DL and prevent coagulation, Labrasol and/or Poloxamer 188 (P188) were added to the SIMVA/DL dispersion.

Specifically, 10 mg of SIMVA and 15 mg of Labrasol were dispersed in 10 mL of DW (SIMVA/Labrasol), and the dispersion was added dropwise to 10 ml of a DL solution (2.88 mg/mL) by continuous vortex mixing to obtain SIMVA/DL/Labrasol.

20 mg of P188 was dissolved in 10 ml of deionized water (DW), and 10 mg SIMVA was added into the tube, with continuous stirring. Separately, 10 mL of the DL aqueous solution (2.88 mg/mL) was added dropwise to the SIMVA/P188 dispersion to obtain SIMVA/DL/P188. 15 mg of Labrasol was added to the SIMVA/DL/P188 dispersion by vortex mixing to obtain SIMVA/DL-CD.

Separately, 10 mg of SIMVA was dispersed in 10 mL of DW including Labrasol (15 mg) and P188 (20 mg) to obtain SIMVA-CD.

All formulations were freeze-dried at −70° C. to completely remove water.

In order to improve in vivo oral bioavailability, water solubility and ASBT-mediated intestinal membrane permeability were improved by incorporating SIMVA into DL micelles, and thus SIMVA incorporation into hydrophobic cores of the DL micelles having weight ratios of 1:1.40 (SIMVA/DL #1), 1:2.88 (SIMVA/DL #2), and 1:5.76 (SIMVA/DL #3) were confirmed in solubility and permeability of SIMVA in artificial intestinal membranes. In particular, solubility and effective permeability (Pₑ) of SIMVA/DL #2 and #3 were higher than those of SIMVA/DL #1 by 309% and 321%, and 1.63 times and 1.92 times, respectively (Table 1). However, an additional increase in the weight ratio of DL to SIMVA (i.e., SIMVA/DL #3) did not further improve permeability.

Subsequently, in order to further improve water dispersibility and permeability of SIMVA/DL, Labrasol and P188, as dispersants, were sequentially added to SIMVA/DL #2, and the weight ratio thereof was optimized.

As a result, after introducing Labrasol, SIMVA/DL/Labrasol #2 and #3 exhibited improved solubility and Pₑ compared to SIMVA/DL #2 by 201% and 213%, and 1.35 times and 1.74 times, respectively. In addition, after introducing P188, SIMVA/DL-CD #2 had Pₑ improved compared to SIMVA/DL/Labrasol #2 by 3.37 times (Table 1). Therefore, the optimum weight ratio of SIMVA, DL, Labrasol, and P188 was determined as 1:2.88:1.5:2 in SIMVA/DL-CD.

TABLE 1
Composition, solubility, and effective permeability
of SIMVA/DL, SIMVA/DL/Labrasol, and SIMVA/DL-CD

| | | | | | | Effective |
|---|---|---|---|---|---|---|
| | | | | | | permeability |
| | SIMVA | DL | Labrasol | P188 | Solubility | (Pₑ, ×10⁻⁶ |
| Formulation | (mg) | (mg) | (mg) | (mg) | (μg/mL) | cm/s) |
| SIMVA | 10 | | | | 0.768 ± 0.115 | ND |
| SIMVA/DL#1 | 10 | 14.4 | | | 23.8 ± 5.12 | 5.52 ± 2.08 |
| SIMVA/DL#2 | 10 | 28.8 | | | 73.6 ± 0.962 | 9.05 ± 3.93 |
| SIMVA/DL#3 | 10 | 57.6 | | | 76.2 ± 2.50 | 10.6 ± 1.72 |
| SIMVA/DL/Labrasol#1 | 10 | 28.8 | 5 | | 82 ± 4.38 | 8.76 ± 1.26 |
| SIMVA/DL/Labrasol#2 | 10 | 28.8 | 15 | | 148 ± 8.65 | 12.3 ± 0.69 |
| SIMVA/DL/Labrasol#3 | 10 | 28.8 | 30 | | 157 ± 2.85 | 15.8 ± 3.62 |
| SIMVA/DL-CD#1 | 10 | 28.8 | 15 | 10 | 169 ± 12.8 | 22.6 ± 4.32 |
| SIMVA/DL-CD#2 | 10 | 28.8 | 15 | 20 | 308 ± 2.95 | 41.5 ± 5.82 |
| SIMVA/DL-CD#3 | 10 | 28.8 | 15 | 40 | 315 ± 5.10 | 46.8 ± 6.65 |

Effective permeability (Pₑ) refers to permeability through artificial intestinal membranes. SIMVA: simvastatin, DL: N^{α}-deoxycholyl-I-lysyl-methylester, P188: Poloxamer 188, CD: colloidal dispersion of Labrasol and P188, SIMVA/DL: solid dispersion of SIMVA and DL, SIMVA/DL-CD: SIMVA/DL-carrying CD, and ND: not detected. Each value indicates average±SD (n=6).

TABLE 2
Particle size, polydispersity index, and zeta potential
of SIMVA using DL, Labrasol, and P188 or SIMVA

| | Particle | Polydispersity | Zeta potential |
|---|---|---|---|
| Test material | size (nm) | index (PDI) | (mV) |
| SIMVA in water | 5721 ± 1655 | 0.95 ± 0.05 | −24.9 ± 0.65 |
| SIMVA in 5% DMSO | 4505 ± 902 | 1.00 ± 0.00 | −5.56 ± 1.04 |
| SIMVA/Labrasol | 175 ± 1.42 | 0.06 ± 0.01 | −11.5 ± 1.02 |
| SIMVA/P188 | 1600 ± 139 | 1.00 ± 0.00 | −14.5 ± 0.65 |
| SIMVA/DL | 2063 ± 288 | 0.19 ± 0.09 | 78.3 ± 4.17 |
| SIMVA-CD | 188 ± 2.52 | 0.12 ± 0.01 | −10.6 ± 1.65 |
| SIMVA/DL/Labrasol | 130 ± 41.3 | 0.39 ± 0.06 | 62.4 ± 2.07 |
| SIMVA/DL/P188 | 150 ± 4.84 | 0.38 ± 0.00 | 46.2 ± 0.26 |
| SIMVA/DL-CD | 125 ± 1.78 | 0.13 ± 0.02 | 64.6 ± 5.69 |

SIMVA: simvastatin, DL: N^{α}-deoxycholyl-I-lysyl-methylester, P188: Poloxamer 188, DMSO: dimethyl sulfoxide, CD: colloidal dispersion of Labrasol and P188, SIMVA/DL: solid dispersion of SIMVA and DL, SIMVA-CD: SIMVA-carrying CD, and SIMVA/DL-CD: SIMVA/DL-carrying CD. Each value indicates average+SD (n=4).

Subsequently, in order to identify formation of a solid dispersion of SIMVA and DL, SIMVA, DL, P188, SIMVA/DL, SIMVA/P188, SIMVA-CD, SIMVA/DL-CD, and a physical mixture of SIMVA, DL, and P188 were analyzed by powder X-ray diffraction (PXRD) and differential scanning calorimetry (DSC) using a D8 Advance diffractometer (Bruker AXS GmbH, Karlsruhe, Germany) and a differential scanning calorimeter (DSC) Q1000 V9.9 Build 303 (TA Instrument Inc., New Castle, DE, USA). In addition, average particle size, polydispersity (PDI), and zeta potential of a waterborne dispersion (aqueous solution) of SIMVA/DL-CD were measured using a dynamic light scattering (DLS) analyzer (Malvern Zetasizer Nano ZS90; Malvern Instruments, Malvern, UK). For morphological evaluation, a high-resolution TEM (HRTEM; JEM-200; JEOL, Tokyo, Japan) was used to observe dispersion of SIMVA/DL-CD.

As a result of identifying structural behaviors of SIMVA in formulations for oral administration using DL, Labrasol, and P188 based on analyses via PXRD and DSC, pure SIMVA exhibited distinguishable crystalline peaks, particularly in a 20 range of the PXRD spectrum as shown in FIG. 1A. Slightly decreased heights of the peaks of the physical mixture of SIMVA, DL, and P188 indicate that SIMVA still has a crystalline structure in the physical mixture. In contrast, crystalline diffraction was lost in the solid SIMVA/DL-CD indicating that solid SIMVA/DL-CD has an amorphous structure. In a thermal analysis of pure SIMVA, a sharp and narrow endothermic peak was observed at 141° C., indicating that pure SIMVA has a crystalline structure (FIG. 1B). However, this property almost disappeared in the thermal analyses of SIMVA/P188, SIMVA/DL, SIMVA-CD, and SIMVA/DL-CD. In addition, the clear endothermic peak of SIMVA was not observed in a thermogram of the physical mixture of SIMVA, DL, and P188, indicating that the structure of SIMVA was converted into an amorphous structure and then molecularly dispersed, and thus this may be derived in the formation of the solid dispersion while heating.

Based on the above-described results, it can be seen that SIMVA of SIMVA/DL-CD may exist in an amorphous state.

In addition, successful incorporation of SIMVA into SIMVA/DL-CD was confirmed by analysis results of particle size, polydispersity (PDI), and zeta potential (Table 2).

The particle size of SIMVA in Labrasol, P188, and DL was smaller than of that of SIMVA in water by 32.6 times, 3.57 times, and 2.77 times, respectively. These results indicate that SIMVA was successfully incorporated into hydrophobic cores of micelles formed by individual surfactants. Optimum particle size and PDI of SIMVA/DL-CD were 125 nm±1.78 nm and 0.13±0.02, which were lower than those of SIMVA/Labrasol, SIMVA/P188, and SIMVA/DL by 1.40 times, 12.8 times, and 16.5 times, respectively.

After incorporation into DL, a positive zeta potential indicates that a hydrophobic DA part was assembled to a lipophilic core, and a hydrophilic and positively charged L-lysine part was located in an aqueous phase in a micelle (FIG. 1C). However, it was assumed that a decrease (1.2 times) in the zeta potential observed in SIMVA/DL-CD, compared to SIMVA/DL, was caused by interaction between a polar head of negatively charged Labrasol and the L-lysine part of DL in SIMVA/DL-CD.

In addition, in transmission electron microscope (TEM) images of SIMVA/DL-CD, formation of spherical objects with a uniform diameter less than 100 nm was observed (FIG. 1D). It was assumed that a difference in particle size of SIMVA/DL-CD micelles between dynamic laser light scattering (DLS) and TEM was caused since loosely aligned micelles, unlike DLS samples, were in contact in a dry state in preparation of TEM samples.

### Example 2: Analysis of Solubility and In Vitro Permeability of SIMVA/DL-CD

### 2-1. Determining Loading Concentration

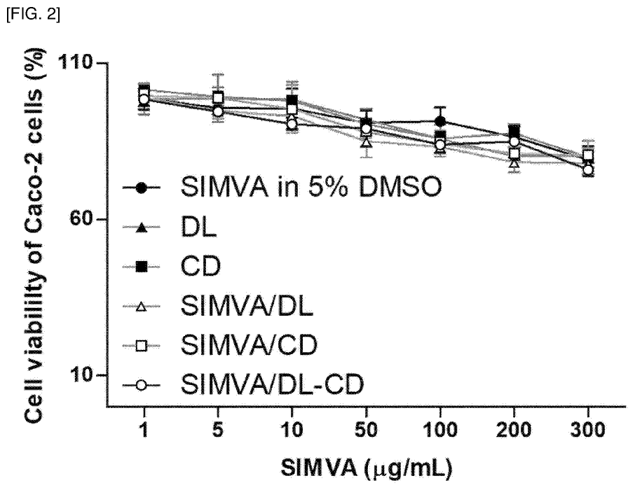

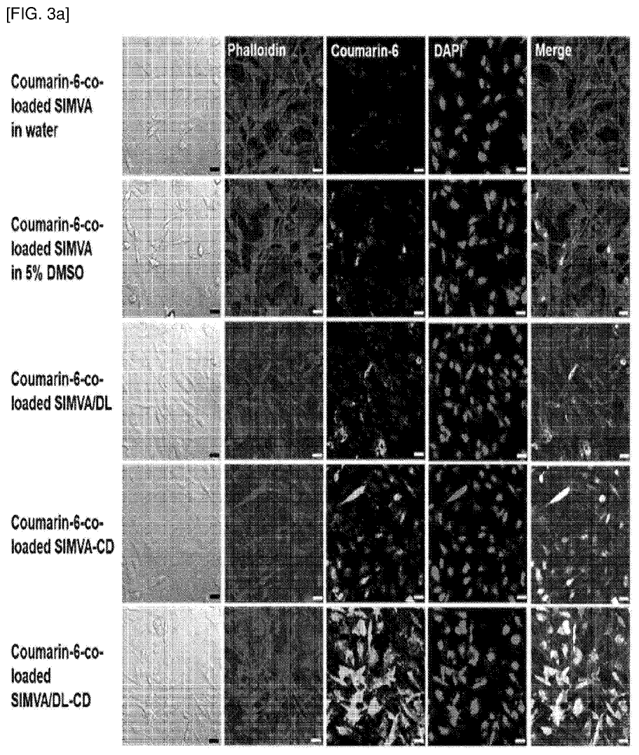

As a result of treating Caco-2 (ATCC® HTB-37™; American Type Culture Collection, Manassas, VA) cells with SIMVA in a concentration of 300 μg/mL for 6 hours, it was confirmed that all SIMVA formulations were non-toxic to the Caco-2 cells (FIG. 2). Based thereon, a loading concentration of SIMVA for analysis of in vitro intestinal tract cell membrane permeability was set equally as 100 μg/mL.

### 2-2. Solubility Analysis

In order to identify improvement of solubility after forming SIMVA/DL-CD, a drug and a carrier were accurately weighed in a predetermined drug-carrier ratio and added to 4 mL of water contained in a screw cap bottle. The prepared mixture was stirred at 25° C. for 24 hours using an orbital shaker. A SIMVA DW dispersion was used as a control. After 24 hours, each sample was centrifuged and filtered, followed by dilution 5 times, and then dissolved SIMVA was measured at room temperature by HPLC using a C18 column (4.6 mm×250 mm, 5 μm, 100 Å). A mobile phase included 0.1% acetonitrile-phosphoric acid (65:35, v/v) in DW, and a flow rate was optimized to 1 mL/min. An injection volume was 20 μL, and measurement was performed at 238 nm using a UV detector.

As a result, in the artificial intestinal tract membrane, P188, Labrasol, and DL respectively improved solubility and effective permeability (Pₑ) of SIMVA. However, SIMVA/DL showed improved solubility compared to SIMVA/P188 and SIMVA/Labrasol by 43.8 times and 50.0 times and enhanced permeability by 200% and 275% (Table 3).

This was assumed because hydrophobic SIMVA was included in the cores of self-assembled micelles formed by DL. However, when P188 and Labrasol were incorporated into SIMVA/DL-CD, the incorporated composite had improved solubility and Pₑ of SIMVA compared to SIMVA/DL by 418% and 4.58 times. In addition, the size of SIMVA/DL-CD was smaller than that of SIMVA/DL by 15.8 times. The size reduction to a nano-scale may be caused by binding to Labrasol and P188, and accordingly absorption of SIMVA may further be increased (Table 3).

In addition, when compared with SIMVA/DL/Labrasol, no solubility-permeability tradeoff was observed in Pₑ of SIMVA/DL-CD, solubility and permeability of SIMVA/DL-CD were improved compared to those of SIMVA/DL/Labrasol by 208% and 404%. This is because of an amorphous property of SIMVA/DL-CD. Therefore, the enhancement in solubility did not decrease the free fraction of drug, with permeability remaining unaffected and a solubility-dependent increment being observed at the artificial membrane. In addition to the improved solubility and permeability into intestinal tract membranes, it was analyzed that positively charged SIMVA/DL-CD permeates more into negatively charged artificial phospholipid membranes due to interaction between charges.

### 2-3. Permeability Analysis

In vitro artificial intestinal tract membrane permeability of SIMVA in water, SIMVA in 5% DMSO, SIMVA/DL, SIMVA/Labrasol, SIMVA/P188, SIMVA-CD, SIMVA/DL/P188, SIMVA/DL/Labrasol, and SIMVA/DL-CD was analyzed by parallel artificial intestinal tract membrane permeability assay (PAMPA; BD Biosciences).

Specifically, 200 μL of a solution of SIMVA (200 μg/mL) in phosphate-buffered saline (PBS, pH 6.8) was added to each well of a donor compartment and 300 μL of PBS (pH 6.8) was added to a receptor compartment. After assembling the receptor and donor plates, incubation was performed at room temperature for 5 hours. After incubation, samples were recovered from the receptor and donor plates, and concentrations of SIMVA were measured using an HPLC system including a UV detector. Pₑ of each drug was calculated using the following equation.

$${P}_{e} = - ln [ 1 - \frac{{C}_{A} ( t )}{{C}_{eq}} ] / [ A \times ( \frac{1}{{V}_{D}} + \frac{1}{{V}_{A}} ) \times t ] \qquad ( 1 )$$

Here, Pₑ: effective permeability (cm/s), A: effective membrane area (0.228 cm²), V_{D}: donor well volume (0.2 mL), VA: receptor well volume (0.3 mL), t: total incubation time(s), and C_{A} (t): concentration of drug in a receptor well at time t, and

$${C}_{eq} = [ {C}_{D} ( t ) \times {V}_{D} + {C}_{A} ( t ) \times {V}_{A} ] / ( {V}_{D} + {V}_{A} ) \qquad ( 2 )$$

Here, C_{D} (t) is a concentration of a drug in a donor well at time t.

In addition, apparent permeability (Pₐₚₚ) of SIMVA/DL-CD in Caco-2/HT-29-MTX-E12 cell membranes was evaluated and shown in Table 3.

Specifically, in vitro apparent permeability (Pₐₚₚ) of SIMVA in water, SIMVA in 5% DMSO, SIMVA/DL, SIMVA/Labrasol, SIMVA/P188, SIMVA-CD, SIMVA/DL/P188, SIMVA/DL/Labrasol, and SIMVA/DL-CD was identified using monolayers of Caco-2 cells and HT29-MTX-E12 cells (EACC 12040401; Public Health England, Oxford, UK). Caco-2 and HT29-MTX-E12 cells were seeded in a 24-well Transwell® filter insert at a concentration of 1×10⁵ cells/well and cultured in Dulbecco's modified Eagle's medium (DMEM) supplemented with 10% (v/v) fetal bovine serum (FBS) and 1% penicillin/streptomycin to form a cell monolayer. In order to further investigate intracellular transport of SIMVA through cell membranes of the Caco-2/HT29-MTX-E12 cells, 0.1 mL of the drug dispersed in HBSS (corresponding to 100 μg/mL SIMVA) and 0.6 mL of HBSS were respectively added to apical and basolateral compartments. At 0.5, 1, 2, 3, 4, and 5 hours, 100 μL of each sample was collected from each basolateral compartment and replaced with the same volume of HBSS. Permeated SIMVA of each sample was measured using an HPLC system equipped with a UV detector, and Pₐₚₚ of SIMVA was calculated using the following equation.

$${P}_{app} = dQ / dt \times 1 / ( A \times {C}_{0} ) \qquad ( 3 )$$

Here, dQ/dt is a linear appearance rate (μmol/s) of a drug in the basolateral compartment, Co is an initial concentration of donor SIMVA (μg/mL), and A is surface area of the monolayer (cm²).

As a result, in SIMVA/DL/Labrasol and SIMVA/DL-CD, after adding Labrasol and P188 thereto, solubility was improved, and the particle size rapidly decreased to a nano-scale range so that it was confirmed that another mechanism (endocytosis, micropinocytosis, or the like) of permeating the monolayer was preferred (Table 3). Solubility and permeability of SIMVA/DL-CD were improved compared to those of SIMVA/DL/Labrasol by 208% and 404%. This may have been due to the amorphous nature of SIMVA/DL-CD. Therefore, the enhancement in solubility did not decrease the free fraction of drug, with permeability remaining unaffected and a solubility-dependent increment being observed at the artificial membrane.

TABLE 3
Effective permeability (Pₑ) and apparent permeability (Pₐₚₚ)
of SIMVA used alone or in combination with DL, Labrasol, and P188
into artificial intestinal membrane and Caco-2 cell monolayer.

| | Aqueous | Effective | Apparent |
|---|---|---|---|
| | solubility | permeability | permeability |
| | of SIMVA | (Pₑ, ×10⁻⁶ | (Pₐₚₚ, ×10⁻⁶ |
| Test material | (μg/mL) | cm/s) | cm/s) |
| SIMVA in water | 0.768 ± 0.115 | 0.000 ± 0.000 | ND |
| SIMVA in 5% | 1.06 ± 0.136 | 8.87 ± 0.968 | ND |
| DMSO | | | |
| SIMVA/Labrasol | 1.47 ± 0.158 | 3.28 ± 0.574 | ND |
| SIMVA/P188 | 1.68 ± 0.605 | 4.51 ± 1.31 | ND |
| SIMVA/DL | 73.6 ± 0.962 | 9.05 ± 3.93 | ND |
| SIMVA-CD | 6.97 ± 0.810 | 6.29 ± 1.39 | ND |
| SIMVA/DL/Labrasol | 148 ± 8.65 | 12.3 ± 0.691 | 0.150 ± 0.007 |
| SIMVA/DL/P188 | 73.0 ± 0.844 | 10.1 ± 2.22 | ND |
| SIMVA/DL-CD | 308 ± 2.95 | 41.5 ± 18.8 | 0.607 ± 0.012 |

CD: colloidal dispersion including Labrasol and P188, SIMVA/DL: solid dispersion of SIMVA and DL, SIMVA-CD: SIMVA-carrying CD, SIMVA/DL-CD: SIMVA/DL-carrying CD, and ND: not detected. Each value indicates average±SD (n=6).

### 2-4. Analysis of Cellular Uptake

In order to qualitatively analyze cellular uptake of SIMVA/DL-CD into Caco-2 cells, and cellular uptake via an apical sodium-dependent bile acid transporter (ASBT), absorption of coumarin-6-loaded-SIMVA/DL-CD was qualitatively analyzed using ASBT-expressing or non-expressing Madin-Darby canine kidney (MDCK) cells.

Specifically, for analysis of cellular uptake of SIMVA of SIMVA in water, SIMVA in 5% DMSO, SIMVA/DL, SIMVA-CD, and SIMVA/DL-CD, 5×10⁵ Caco-2 cells were inoculated on a cover slip coated with Cell-Tak and cultured until a monolayer was formed. Coumarin-6-loaded SIMVA in water, SIMVA in 5% DMSO, SIMVA/DL, SIMVA-CD, and SIMVA/DL-CD were diluted with DMEM such that the concentration of SIMVA was 100 μg/mL and the cells were treated therewith. After the cells were cultured at 37° C. for 3 hours, actin filaments were stained with phalloidin-rhodamine (100 nM) and nuclei were stained with 4′,6-diamidino-2-phenylindole (DAPI) (1 μg/mL). Then, the cells were analyzed using a confocal laser scanning microscope (Carl Zeiss, Oberkochen, Germany).

In order to investigate absorption of SIMVA/DL-CD via ASBT in the intestinal tract membranes, MDCK cells (ATCC® CCL-34™) were transfected with a gene encoding ASBT using Lipofectamine 2000® and cultured on a 10 mm cover slip coated with a Cell-Tak solution for 20 minutes until a monolayer was formed. The monolayer was treated respectively with 100 μL of coumarin-6-loaded SIMVA in water, SIMVA in 5% DMSO, SIMVA/DL, SIMVA-CD, and SIMVA/DL-CD each having a SIMVA concentration of 100 μg/mL and cultured at 37° C. for 3 hours. Subsequently, the cells were washed with cold HBSS, immobilized with cold 4% paraformaldehyde, blocked using a blocking buffer (0.3% Triton X-100 and 10% normal goat serum in PBS [pH 7.4]), treated with anti-human ASBT antibody (1:500), and cultured overnight. Then, the cells were stained using a secondary antibody labeled with 10 μg/mL Alexa Fluor 546 (Abcam) for 1 hour, and fluorescent images thereof were observed using a confocal laser scanning microscope.

In addition, absorption of SIMVA contained in SIMVA in water or SIMVA in 5% DMSO, SIMVA/DL, SIMVA-CD, and SIMVA/DL-CD, into Caco-2 cells, MDCK cells, and MDCK cells transfected with ASBT was quantified.

Specifically, Caco-2 and MDCK cells were inoculated on a 6-well plate containing DMEM supplemented with 10% FBS and 1% penicillin/streptomycin at a density of 1×10⁵ per well. At the same time, MDCK cells were transfected with the above-described gene encoding ASBT. When a cell confluence reached 80% or more, the culture medium was removed, and the cells were treated with SIMVA in water, SIMVA in 5% DMSO, SIMVA/DL, SIMVA-CD, and SIMVA/DL-CD each having a SIMVA concentration of 100 μg/mL at 37° C. for 1 hour to 3 hours. The cells were washed three times with ice-cold PBS (pH 7.4), treated with trypsin, centrifuged, and collected using 500 μL of acetonitrile. The cell membranes were disrupted using an ultrasonic homogenizer to release the drug contained in the cells, and the concentration of SIMVA contained in a supernatant was measured using LC/MS. Specifically, 100 μL of lovastatin (0.5 μg/mL, internal standard substance) was added to 400 μL of each supernatant, followed by extraction with 4 mL of ethyl acetate:hexane (90:10, v/v). The extracted supernatant was transferred to a glass vial and dried using a rotary evaporator. Finally, the residue was re-dissolved in 100 μL of acetonitrile: DW (80:20, v/v), and the amount of the drug absorbed by each cell type was quantified with an Agilent 6120 quadruple LC/MS system using a Luna C18 column (150 mm×4.6 mm, 5 μm) and a mobile phase (a mixture of acetonitrile and 3 mM formic acid (65:35, v/v)) at a flow rate of 0.8 mL/min. SIMVA and lovastatin were ionized using an atmospheric pressure ionization-electron spray (API-ES) source in a cationic mode under the following conditions: a capillary voltage of 3.0 kV; a drying gas flow rate of 10 L/min; and a drying gas temperature of 300° C., and fragment ions were quantified with respect to SIMVA and lovastatin ([M+H]⁺=441.3) and ([M+H]⁺=405.6).

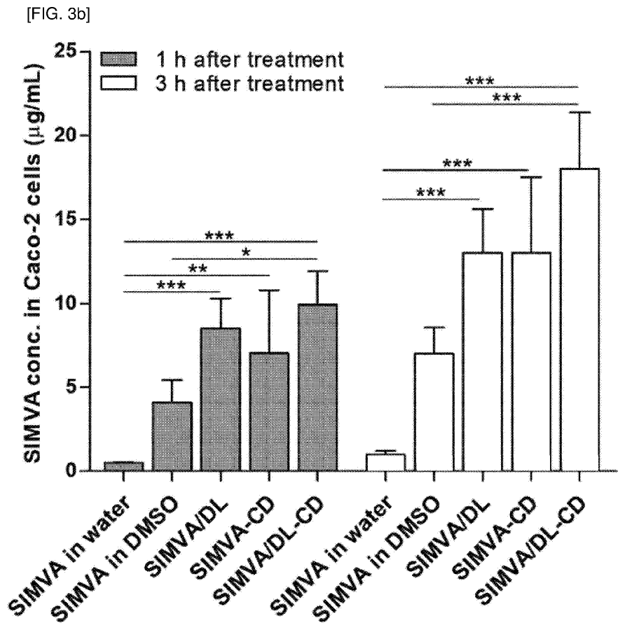

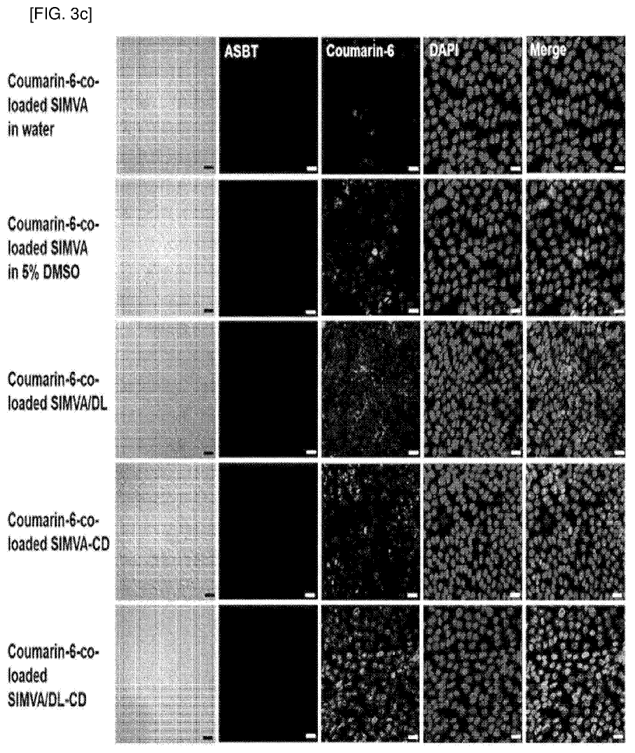

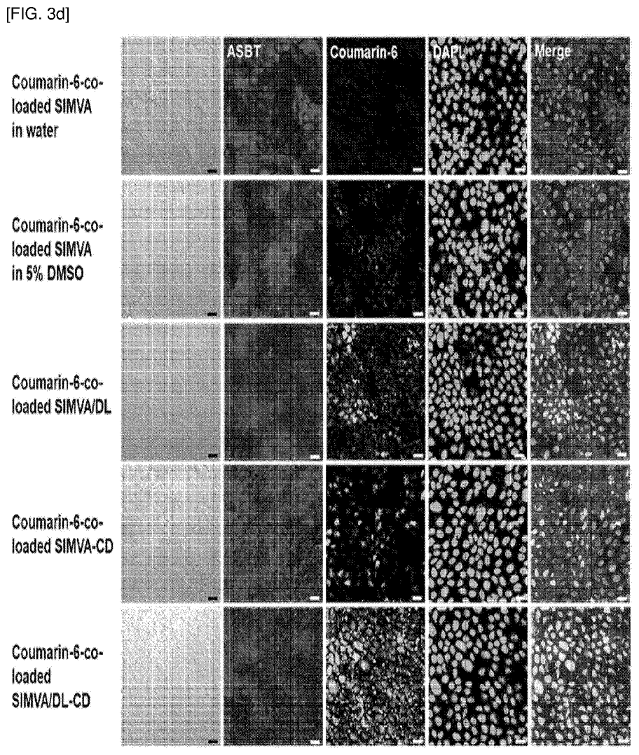

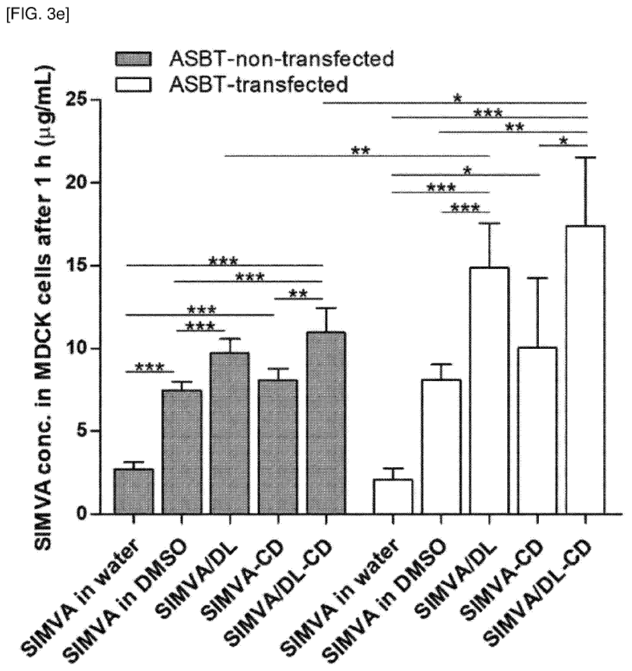

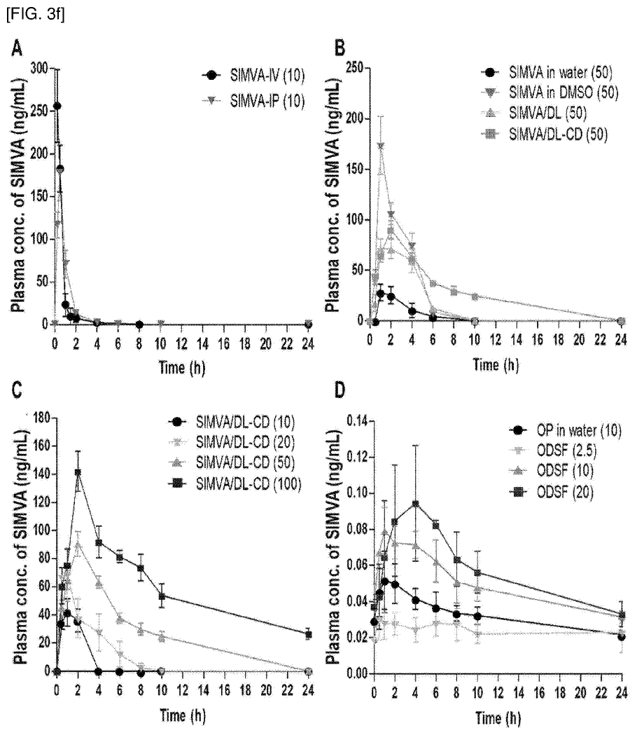

As a result, coumarin-6-loaded SIMVA-CD exhibited more internalization and micelle assembly in nuclei of the cells when compared with coumarin-6-co-loaded SIMVA in water or 5% DMSO (FIG. 3A). After adding DL to SIMVA-CD, it was confirmed that permeation of coumarin-6 into intestinal tract cell membranes in SIMVA/DL-CD was significantly improved when compared with SIMVA/DL and SIMVA-CD.

The cellular uptake of SIMVA by SIMVA-CD were 7.03 μg/mL+3.73 μg/mL and 13.2 μg/mL+4.51 μg/mL after 1 hour and 3 hours, which were higher values than those by SIMVA dispersed in water by 13.5 times and 6.69 times (FIG. 3B). Meanwhile, it was confirmed that exposure of Caco-2 cells to SIMVA/DL-CD for 3 hours further increased cellular uptake of the drug up to 18.6 μg/mL+3.38 μg/mL, which is 9.40-fold and 1.40-fold higher than SIMVA in water and SIMVA/DL-CD at the same time point, respectively. Based thereon, it was confirmed that the improved absorption of SIMVA/DL-CD resulted from ASBT-mediated transport during permeation into the cell membranes.

SIMVA-CD exhibited similar cellular uptake of coumarin-6 regardless of ASBT expression. In contrast, in both SIMVA/DL and SIMVA/DL-CD, cellular uptake of coumarin-6 significantly increased in ASBT-expressing cells compared to ASBT-non-expressing cells (FIGS. 3C and 3D). Furthermore, SIMVA/DL-CD exhibited higher cellular uptake in the ASBT-expressing cells compared to SIMVA-CD, indicating that there were synergistic effects of P188 and Labrasol, and DL on ASBT-promoted cellular uptake.

Similarly, a maximum SIMVA concentration measured after treating the ASBT-non-expressing MDCK cells with SIMVA/DL-CD for 3 hours was 12.7 μg/ml±0.872 μg/mL, which was 1.16-fold and 1.26-fold higher than those of the cells treated with SIMVA/DL and SIMVA-CD (FIGS. 3E and 3F). Furthermore, SIMVA/DL and SIMVA/DL-CD, both including DL, increased cellular uptake in the ASBT-expressing MDCK cells by 210% and 223% after 3 hours compared to the ASBT-non-expressing MDCK cells. That is, in the ASBT-expressing MDCK cells, cellular uptake of SIMVA/DL-CD increased by 9.34 times and 2.19 times compared to SIMVA in water and SIMVA-CD, respectively. Therefore, the quantitative analysis results were consistent with qualitative evaluation results of cellular uptake.

Based on the results, it was confirmed that the structural orientation of DL in SIMVA/DL/Labrasol and SIMVA/DL-CD increased interaction with ASBT, and infiltration of the drug into intestinal tract cell membranes was promoted by ASBT-mediated endocytosis.

### Example 3: In Vivo Oral Absorption of SIMVA/DL-CD in Rats

In order to evaluate effects of DL on solid dispersion, rats were orally administered with free SIMVA, SIMVA/DL, and a formulation of SIMVA/DL-CD with Labrasol and P188, and pharmacokinetic properties thereof were determined.

Specifically, SD rats were randomly divided into 9 groups (4 rats per group). Rats in the first group were intraperitoneally (IP) administered with 400 μL of SIMVA in DMSO (10 mg/kg). Rats of 7 groups were orally administered with 400 μL of SIMVA in water (50) (SIMVA dispersed in water, 50 mg/kg), SIMVA in DMSO (50) (SIMVA dispersed in DMSO, 50 mg/kg), SIMVA/DL (50) (50 mg/kg SIMVA), SIMVA/DL-CD (10) (10 mg/kg SIMVA), SIMVA/DL-CD (20) (20 mg/kg SIMVA), SIMVA/DL-CD (50) (50 mg/kg SIMVA), and SIMVA/DL-CD (100) (100 mg/kg SIMVA). In order to calculate in vivo oral bioavailability, 150 μL of SIMVA (10 mg/kg) dissolved in polyethylene glycol: saline (60:40, v/v) was injected into rats of the other group via the tail vein. Then, 150 μL of blood samples were collected from retroorbital plexus at a predetermined time point. Each blood sample was mixed with 50 μL of a 3.8% sodium citrate solution and centrifuged immediately (2500×g, 15 min, 4° C.). The resultants were stored at −70° C. until plasma samples were obtained and analyzed. The concentration of SIMVA of each plasma sample was analyzed using LC/MS.

After SD rats were orally administered with various concentrations of ODSF repeatedly, the rats were divided into 5 groups (4 rats per group) to evaluate pharmacokinetic properties of OP. The rats were orally administered with a daily dose of 400 μL of OP (10) (OP dispersed in water, 10 mg/kg) or ODSF (2.5), ODSF (10), and ODSF (20) (containing 2.5 mg/kg, 10 mg/kg, and 20 mg/kg OP, respectively), for 14 days. In order to evaluate in vivo oral bioavailability, 150 μL of an aqueous solution of OP (5 mg/kg) was injected via the tail veins of rats of one group at day 1 and day 14 (intravenous administration, IV). Blood samples were collected and analyzed in the same manner as in the SIMVA concentration analysis. Plasma concentrations of OP were measured based on the platinum concentration, after decomposition with nitric acid at 100° C., by inductively coupled plasma-mass spectroscopy (ICP-MS) at a high frequency power of 1450 W, at a coolant flow rate of 12 L/min, at an axillary flow rate of 2.3 L/min, and at a nebulizer flow rate of 0.89 L/min.

As a result, plasma concentration-time curves and pharmacokinetic parameters of IV administration, IP administration, and oral administration of SIMVA, solid dispersion of SIMVA and DL (SIMVA/DL) or SIMVA/DL-CD to the rats are shown in FIGS. 4A to 4C. The pharmacokinetic parameters were estimated using a noncompartmental model of the WinNonlin® 5.3 software (Pharsight Corporation; Sunnyvale, CA, USA), and the calculated pharmacokinetic parameters are shown in Table 4.

TABLE 4
Pharmacokinetic parameters of SIMVA in rats after IV or IP injection of SIMVA or oral administration
of SIMVA, SIMVA/DL, and SIMVA/DL-CD having various concentrations of SIMVA

| | SIMVA- | | SIMVA in | SIMVA in | SIMVA/ | SIMVA/ | SIMVA/ | SIMVA/ | SIMVA/ |
|---|---|---|---|---|---|---|---|---|---|
| Test | IV | SIMVA- | water | DMSO | DL | DL-CD | DL-CD | DL-CD | DL-CD |
| material | (10) | IP (10) | (50) | (50) | (50) | (10) | (20) | (50) | (100) |
| Administration | IV | IP | Oral | Oral | Oral | Oral | Oral | Oral | Oral |
| route | | | | | | | | | |
| Dose of | 10 | 10 | 50 | 50 | 50 | 10 | 20 | 50 | 100 |
| SIMVA | | | | | | | | | |
| (mg/kg) | | | | | | | | | |
| Tₘₐₓ (h) | — | 0.50 ± | 1.50 ± | 1.00 ± | 2.00 ± | 1.00 ± | 0.63 ± | 4.00 ± | 2.00 ± |
| | | 0.00 | 0.58 | 0.00 | 1.73 | 0.00 | 0.25 | 0.00 | 0.00 |
| T_{1/2} (h) | 0.39 ± | 0.54 ± | 1.60 ± | 1.22 ± | 1.47 ± | NE | 1.69 ± | 5.60 ± | 10.4 ± |
| | 0.25 | 0.16 | 0.39 | 0.08 | 0.00 | | 0.30 | 3.43 | 1.18 |
| Cₘₐₓ | 389 ± | 178 ± | 28.7 ± | 174 ± | 75.0 ± | 41.8 ± | 75.5 ± | 90.4 ± | 142 ± |
| (ng/mL) | 202 | 32.9 | 8.59 | 28.8 | 5.25 | 9.71 | 11.1 | 8.71 | 14.2 |
| AUCₗₐₛₜ | 203 ± | 163 ± | 84.0 ± | 469 ± | 300 ± | 66.5 ± | 217 ± | 506 ± | 1409 ± |
| (ng · h/mL) | 44.8 | 13.6 | 28.7 | 15.4 | 18.7 | 13.7 | 46.1 | 8.24 | 66.3 |
| AUC_{inf} | 208 ± | 170 ± | 95.1 ± | 484 ± | 323 ± | NE | 238 ± | 717 ± | 1810 ± |
| (ng · h/mL) | 47.5 | 11.9 | 32.7 | 22.9 | 28.5 | | 51.2 | 168 | 58.2 |
| Bioavailability | 100 | 80.3 ± | 5.34 ± | 46.1 ± | 29.5 ± | 32.7 ± | 53.4 ± | 49.7 ± | 69.3 ± |
| (%) | | 6.70 | 3.19 | 1.51 | 1.84 | 6.74 | 11.3 | 0.811 | 3.26 |

Each value indicates average+SD (n=4).

$$Bioavailability ( \% ) \text{:} ( {AUC}_{last , IP or oral} / {Dose}_{SIMVA , IP or oral} ) / ( {AUC}_{last , IV} / {Dose}_{SIMVA , IV} ) \times 100.$$

Tₘₐₓ: time to reach a maximum plasma concentration, T_{1/2}: half-life of plasma concentration, Cₘₐₓ: maximum plasma concentration, AUCₗₐₛₜ: area under a plasma concentration-time curve from 0 to a last measurable plasma concentration, and AUCinf: area under a plasma concentration-time curve from 0 to infinity.

FIG. 4A shows a plasma concentration-time profile after IV and IP administration at a dose equivalent to 10 mg/kg SIMVA. Bioavailability of SIMVA-IP (10) was 80.3% of that of SIMVA-IV (10) (FIG. 4A and Table 4). In addition, after oral administration of 50 mg/kg SIMVA [SIMVA in DMSO (50)], an achieved maximum plasma concentration (Cₘₐₓ) and an area under the plasma concentration-time curve (0-24 hours) (AUCₗₐₛₜ) were increased compared to SIMVA in water (50) by 6.21 times and by 558%. In vivo oral bioavailability was improved compared to SIMVA in water (50) by 863% (FIG. 4B and Table 4). The improvement of in vivo oral bioavailability of SIMVA in DMSO (50) was analyzed to be due to improved solubility and a high absorption property of DMSO in the intestinal epithelium. SIMVA/DL including 50 mg/kg SIMVA [SIMVA/DL (50)] increased AUCₗₐₛₜ and bioavailability compared to SIMVA in water (50) by 357% and 552%, respectively, and thus effects of DL on improving solubility and permeability consistent to the results of in vitro permeability tests were confirmed. Furthermore, SIMVA/DL-CD including 50 mg/kg SIMVA [SIMVA/DL-CD (50)] for oral administration exhibited higher in vivo oral bioavailability than SIMVA/DL (50) by 168%. Additional improvement of in vivo oral bioavailability of SIMVA in SIMVA/DL-CD re-confirmed synergistic effects of P188 and Labrasol on improving in vivo oral bioavailability of SIMVA/DL via passive diffusion and ASBT-mediated transport. The significant decrease in the micelle size of SIMVA/DL-CD due to the synergistic effects of DL, Labrasol, and P188 may further promote oral absorption via endocytosis and micropinocytosis commonly used by nanoparticles.

In addition, effects of a dose increase on in vivo oral bioavailability of SIMVA/DL-CD including 10 mg/kg, 20 mg/kg, 50 mg/kg, and 100 mg/kg SIMVA [SIMVA/DL-CD (10), SIMVA/DL-CD (20), SIMVA/DL-CD (50), and SIMVA/DL-CD (100)] were confirmed (FIG. 4C and Table 4). In vivo oral bioavailability of SIMVA/DL-CD (10) was higher than that of SIMVA in water (50) by 612%. However, a 2-fold dose increase in SIMVA/DL-CD (20) resulted in a 1.80-fold greater Cₘₐₓ and a 3.23-fold greater AUCₗₐₛₜ, SIMVA/DL-CD (50) exhibited a 2.16-fold greater Cₘₐₓ and a 7.60-fold greater AUCₗₐₛₜ, and SIMVA/DL-CD (100) exhibited a 3.38-fold greater Cₘₐₓ and a 21.1-fold greater AUCₗₐₛₜ compared to those of SIMVA/DL-CD (10). Thus, in vivo oral bioavailability of SIMVA/DL-CD (20, 50, and 100) was improved by 163%, 151%, and 211%, respectively, compared to that of SIMVA/DL-CD (10), indicating an increase in a dose-dependent manner.

Subsequently, it was confirmed that in vivo oral bioavailability of ODSF was significantly improved compared to free OP in a previous study. Plasma profiles of not only OP in water (10 mg/kg) [OP (10)] but also ODSF including 2.5 mg/kg, 10 mg/kg, and 20 mg/kg OP [ODSF (2.5), ODSF (10), and ODSF (20), respectively] were identified after 2-week continuous metronomic administration in comparison with a single IV bolus dose of 5 mg/kg OP [OP-IV (5)] administered on day 1 and day 14.

ODSF, which is an ionic composite of oxaliplatin (OP) and DL, was prepared by dissolving 10 mg of OP in a P188 solution (10 mg/mL) in DW including 75 μL of Labrasol. After completely solubilizing OP, 10 mL of a DL solution (2.88 mg/mL in DW) was added thereto at a molar ratio of 1:2 to form an ionic composite, followed by freeze-drying at −70° C.

As a result, as shown in FIG. 4D and Table 5, after 13 days of repeated oral administration of OP or ODSF, a plasma concentration of the drug was maintained at the oral dose of OP.

TABLE 5
Pharmacokinetic parameters of OP in rats on day 14 after second IV injection
of OP and oral administration of OP or various doses of ODSF once a day

| Test material | OP | OP (10) | ODSF (2.5) | ODSF (10) | ODSF (20) |
|---|---|---|---|---|---|
| Administration | IV | Oral | Oral | Oral | Oral |
| route | | | | | |
| Dose of OP | 5 | 10 | 2.5 | 10 | 20 |
| (mg/kg) | | | | | |
| T_{max, on day 14} (h) | — | 0.833 ± 0.289 | 8.25 ± 11.1 | 1.33 ± 0.58 | 4.47 ± 1.16 |
| T_{1/2, on day 14} (h) | 15.8 ± 1.32 | 23.7 ± 0.723 | 30.4 ± 12.3 | 23.6 ± 7.37 | 17.8 ± 1.79 |
| C_{max, on day 14} | 1.06 ± 0.337 | 0.053 ± 0.014 | 0.038 ± 0.009 | 0.079 ± 0.009 | 0.101 ± 0.023 |
| (μg/mL) | | | | | |
| AUC_{last, on day 14} | 5.46 ± 1.46 | 0.775 ± 0.124 | 0.579 ± 0.107 | 1.17 ± 0.146 | 1.38 ± 0.314 |
| (μg · h/mL) | | | | | |
| AUC_{inf, on day 14} | 8.12 ± 2.47 | 1.50 ± 0.196 | 1.35 ± 0.450 | 2.22 ± 0.085 | 2.22 ± 0.463 |
| (μg · h/mL) | | | | | |
| Bioavailability | 100 | 7.09 ± 1.14 | 21.2 ± 3.93 | 10.7 ± 1.34 | 6.30 ± 1.44 |
| (%) | | | | | |

Each value was expressed as average±SD (n=4). OP/DL: ion-pair composite of OP and DL.

In addition, the plasma concentrations of all OP formulations returned to initial levels on day 14 of oral administration. In the normal state, bioavailability after oral administration of ODSF (2.5) was 21.2%+3.93%, which was higher than OP (10) by 299%, and bioavailability after oral administration of ODSF (10) was 1.5-fold higher than that of free OP. In addition, AUCₗₐₛₜ and Cₘₐₓ of ODSF (10) were higher than those of ODSF (2.5) by 207% and 2.02 times on day 14. Similarly, a 2-fold dose increase in ODSF, i.e., 20 mg/kg OP, exhibited a Cₘₐₓ of 0.101 μg/mL+0.023 μg/mL and an AUCₗₐₛₜ of 1.38 μg·h/mL+0.14 μg·h/mL on day 14, which were higher than those of ODSF (10) by 127% and 1.17 times.

Therefore, it was confirmed that oral absorption of ODSF increased in an OP dose-dependent manner in the range of 2.5 mg/kg to 20 mg/kg.

### Example 4: In Vitro Cytotoxicity and Adjuvant Immunotherapy Effect of SIMVA/DL-CD

Considering that SIMVA easily binds to albumin in plasma, SIMVA bound to albumin cannot reach a concentration sufficient for inducing apoptosis of tumor cells due to a low absorption rate thereof. Thus, in order to investigate in vitro cytotoxicity of SIMVA, DL, CD of Labrasol and P188 (CD), SIMVA/DL, CD of SIMVA including Labrasol and P188 (SIMVA-CD), and SIMVA/DL-CD in various concentrations of SIMVA from 1 μg/mL to 200 μg/mL, viability of CT26 cells were analyzed using cell counting kit-8 (CCK-8, Dojindo Molecular Technologies, Rockville, MD, USA).

Specifically, 5×10³ CT26 cells were inoculated on 100 μL of DMEM and a Roswell Park Memorial Institute (RPMI) medium, respectively supplemented with 10% (v/v) FBS and 1% penicillin/streptomycin. After culturing the cells at 37° C. for 24 hours, the cells were cultured with 100 μL of SIMVA, SIMVA/DL, SIMVA-CD, and SIMVA/DL-CD (1 μg/mL, 5 μg/mL, 10 μg/mL, 50 μg/mL, 100 μg/mL, 200 μg/mL, and 300 μg/mL SIMVA). DL and CD were also treated in the same volume and each of the concentrations. After 24 hours of the culturing, 10 μL of a WST-8 solution [2-2-methoxy-4-nitrophenyl)-3-(4-nitrophenyl)-5-(2,4-disulfophenyl)-2H-tetrazolium monosodium salt] was diluted with 100 μL of each medium, followed by culturing for 2 hours. Then, absorbance was measured using a microplate reader (PerkinElmer multimode plate reader) at 450 nm. Percentages of viable cells are shown by comparing the number of treated cells with that of untreated cells.

Next, in order to evaluate apoptosis of CT26 cells induced by simvastatin regardless of serum, Annexin V-Propidium iodide (PI) analysis was performed.

Specifically, CT26 cells were inoculated on a 6-well plate containing an RPMI 1640 medium supplemented with 10% FBS and 1% antibiotic-antifungal agent at a density of 1×10⁵ and cultured at 37° C. for 24 hours. Then, the cells were treated with 20 UM SIMVA or SIMVA/DL-CD (equivalent to 20 M SIMVA) in an RPMI 1640 medium supplemented with 1% FBS or without FBS. After 24 of the culturing, the cells were washed and resuspended with an annexin binding buffer, and annexin V and PI were added thereto. After staining for 15 minutes, samples were analyzed by flow cytometry.

As a result, as shown in FIG. 5A, cell viabilities of 80% or more were confirmed in the cells treated with DL and CD in a SIMVA concentration range of 1 μg/mL to 150 μg/mL, and the cell viabilities were maintained at 77.1% and 82.1% at the maximum SIMVA concentration of 200 μg/mL.

IC₅₀, defined as a concentration of a drug required to kill 50% of cells during a predetermined period, was 183 μg/mL±1.52 μg/mL in the case of SIMVA dispersed in water. While SIMVA/DL and SIMVA-CD exhibited IC₅₀ values of 28.3 μg/ml±0.857 μg/mL and 102 μg/mL±1.11 μg/mL, respectively, SIMVA/DL-CD exhibited an IC₅₀ value of 3.47 μg/mL±0.299 μg/mL, which is lower than those of SIMVA/DL and SIMVA-CD by 83% and 290%, respectively.

Also, in PI analysis, SIMVA/DL-CD exhibited superior cytotoxicity to SIMVA under 0% FBS and 1% FBS conditions (FIG. 5B).

Accordingly, it can be seen that cancer cell viability may be significantly reduced by improving solubility and permeability of SIMVA even in the presence of serum.

### Example 5: In Vivo Antitumor Effect of SIMVA/DL-CD

In order to identify antitumor effects of SIMVA/DL-CD, a syngeneic mouse model was prepared by inoculating 1×10⁶ CT26 cells suspended in 100 μL PBS (pH 7.4) on the right side of the back of each mouse. When a tumor volume reached 50 mm³ to 70 mm³, the mice were randomly divided into 7 treated groups (10 mice per group) and administered as follows every day for 18 days (FIG. 6A): Control (untreated), SIMVA-IP (20) (20 mg/kg SIMVA, once a day, IP administration), SIMVA in water (20) (20 mg/kg SIMVA dispersed in water, once-a-day, oral administration), SIMVA in DMSO (20) (20 mg/kg SIMVA dispersed in DMSO, once-a-day, oral administration), SIMVA/DL-CD (10) (SIMVA/DL-CD dispersed in water, 10 mg/kg SIMVA, once-a-day, oral administration), SIMVA/DL-CD (20) (SIMVA/DL-CD dispersed in water, 20 mg/kg SIMVA, once-a-day, oral administration) and SIMVA/DL-CD (50) (SIMVA/DL-CD dispersed in water, 50 mg/kg SIMVA, once-a-day, oral administration). Tumor size and body weight were measured every 3 days in all groups. The tumor volume was calculated using the following equation, and therapeutic effects on tumor regression were determined.

$$tumor volume = {a}^{2} \times b \times 0.52 \qquad ( 3 )$$

Here, a is tumor width and b is tumor length.

In addition, on day 18 of administration, the mice were sacrificed and the tumor weight measured.

As a result, SIMVA in water (20) did not exhibit anticancer effects, SIMVA/DL-CD (10) delayed the tumor growth and decreased the weight of the isolated tumors more than the control (untreated) by 154% and 145%, respectively (FIGS. 6B and 7A). Significantly greater tumor suppression capability of SIMVA/DL-CD (10) even at a low dose was assumed because a plasma drug level required to induce anticancer response was achieved due to improved oral absorption by SIMVA/DL-CD.

SIMVA/DL-CD (20) delayed the tumor growth more than SIMVA in DMSO (20) by 1.92 times and more than SIMVA-IP (20) by 2.46 times and reduced the weight of tumors more than SIMVA-IP (20) and SIMVA in DMSO (20) by 361% and 272%, respectively, without toxicity (FIGS. 6B and 6C). However, anticancer effects of SIMVA/DL-CD (50) were similar to those of SIMVA/DL-CD (20) and did not show plasma drug concentration-dependent improvement in the tumor suppressing effects (FIG. 6B, FIG. 7A, and Table 4).

These results indicate that SIMVA/DL-CD may effectively deliver SIMVA via an oral administration route and exhibit far greater antitumor effects than the other solubilizers or administration routes.

Thereafter, an optimal dose of SIMVA/DL-CD for oral administration to induce antitumor immunomodulatory effects in a CT26 mouse colon cancer model was identified. SIMVA/DL-CD was orally administered in doses of 10 mg/kg, 20 mg/kg, and 50 mg/kg according to a schedule shown in FIG. 7A.

As a result, in all doses, the growth of tumors was significantly delayed without toxicity (FIGS. 6D and 7A). For flow cytometry, after isolating tumors, tumor single cell suspension was prepared using a tumor dissociation kit (MACS), and then erythrocytes were removed using an RBC lysis buffer, and dead cells were removed using a dead cell removal kit. The single cell suspension was pre-blocked using a FcR antibody and labeled with a fluorescent-conjugated antibody at a predetermined ratio in accordance with the manufacturer's instructions. Samples were analyzed by flow cytometry, and data thereof was analyzed using FlowJo software (Tree Star). Oral administration of SIMVA/DL-CD increased a proportion of immune cells in TMEs (FIGS. 7B and 8). Although the increase in CD8⁺ T cells, among the immune cell types, was significant at boundaries as the dose increased, the percentage of CD8⁺ T cells expressing cytotoxic CD107α remarkably increased due to administration of SIMVA/DL-CD (20) and SIMVA/DL-CD (50) (FIG. 7C). After administering SIMVA/DL-CD, no difference was observed between CD4⁺ T cell population and regulatory T cell (T_{reg}) population (FIG. 6E). Myeloid-derived suppressor cells (MDSCs) and M2-like macrophages, serving as immunosuppressive cells, tended to decrease as the dose of SIMVA/DL-CD increased (FIG. 7D).

Based thereon, it was confirmed that SIMVA/DL-CD (20) induced an antitumor immune response similar to that induced by SIMVA/DL-CD (50) and significantly suppressed proliferation of tumor cells via immunoregulatory effects without toxicity, and thus SIMVA/DL-CD (20) was selected at an optimal dose for co-administration with ODSF.

### Example 6: In Vivo Antitumor Effect of SIMVA/DL-CD and ODSF

6-1. Effect of Combination therapy on CT26.CL25 Tumor Expressing Immunogenic Neoantigen β-Galactosidase

Metronomic oral administration of a multiple nanoemulsion loaded with OP/DL exhibited immunoregulatory effects including an increase in Dendritic cell (DC) function. Based on SIMVA inducing antigen presentation of DCs as well as tumor cell apoptosis (FIG. 7B), it was expected that a combination of SIMVA/DL-CD and ODSF would induce a strong antitumor immune response.

To confirm this, antitumor effects of combination therapy were investigated in CT26.CL25 tumors expressing immunogenic neoantigen-galactosidase. SIMVA/DL-CD (10) and ODSF (10) were orally administrated in accordance with a schedule shown in FIG. 9A, and the mice were sacrificed on day 18 to evaluate effects on immune population. Administration of SIMVA/DL-CD (10) or ODSF (10) alone slightly delayed tumor growth and reduced tumor weight (FIG. 9B). However, the combination of SIMVA/DL-CD (10) and ODSF (10) exhibited far superior antitumor effects compared to single therapy (FIG. 9B). No significant weight change was observed in the treated mice (FIG. 9C). Also, the combination therapy increased the CD8⁺ T cell population and reduced immunosuppressive T_{reg} and MDSC in TME (FIG. 9D).

As expected, the delivery system applied to OP improved bioavailability (FIG. 4D), and thus oral administration of ODSF corresponding to 10 mg/kg OP [ODSF (10)] delayed tumor growth without weight loss in the tumor model.

### 6-2. Preparation of Animal Model

In order to analyze in vivo antitumor effects of orally administered SIMVA/DL-CD, ODSF, and a combination thereof, a syngeneic mouse model was prepared by inoculating 1×10⁶ CT26 cells on a side of each mouse. When a tumor volume reached 50 mm³ to 70 mm³, the mice were randomly divided into 4 groups (10 mice per group) and administered as follows every day for 18 days: Control (untreated), SIMVA/DL-CD (20) (containing 20 mg/kg SIMVA, once-a-day, oral administration), ODSF (10) (containing 10 mg/kg OP, once-a-day, oral administration), and SIMVA/DL-CD (20)+ODSF (10) (each once-a-day, oral administration). Tumor size and body weight were measured every 3 days in all groups. After treatment, the mice were sacrificed, and tumors were harvested for additional analysis. In addition, stomach, jejunum, liver, spleen, kidney, and lung of each mouse were excised and immobilized in a 4% formalin solution, followed by H&E staining to evaluate toxicity by SIMVA/DL-CD and ODSF.

In order to analyze antitumor effects of oral administration of 10 mg/kg SIMVA [SIMVA/DL-CD (10)] or 10 mg/kg ODSF [ODSF (10)] alone, or a combination thereof [SIMVA/DL-CD (10)+ODSF (10)], male BALB/c mice inoculated with 1×10⁶ CT26.CL25 cells were prepared. When an average tumor size reached 50 mm³ to 70 mm³, the mice having tumors were administered with SIMVA/DL-CD (10), ODSF (10), or SIMVA/DL-CD (10)+ODSF (10) and tumor size and body weight were measured every 3 days in all groups.

In addition, in order to analyze in vivo antitumor effects of SIMVA/DL-CD and ODSF together with anti-programmed cell death protein-1 (αPD-1), 1×10⁶ CT26 cells were inoculated on the left side of each male BALB/c mouse. On day 6 after inoculation of the tumor cells, each mouse was administered with αPD-1, SIMVA/DL-CD (20)+ODSF (10), or αPD-1+SIMVA/DL-CD (20)+ODSF (10). All formulations were orally administered once a day, and αPD-1 was intraperitoneally injected. The tumor size was measured every 3 days using a caliper. In order to evaluate immunological memory formation, several mice exhibiting complete tumor suppression in the group treated with αPD-1+SIMVA/DL-CD (20)+ODSF (10) were rechallenged on the opposite side with the same tumor model.

### 6-3. Immunofluorescence Staining

CT26 tumor cell-bearing mice were administered with SIMVA/DL-CD (20) (containing 20 mg/kg SIMVA, once-a-day, oral administration), ODSF (10) (containing 10 mg/kg OP, once-a-day, oral administration), and SIMVA/DL-CD (20)+ODSF (10) (each once-a-day, oral administration). On day 21, the mice were sacrificed, and tumor tissue was collected to prepare a formalin-fixed paraffin-embedded tissue slide. In the case of CD8 staining, a sample slide was deparaffinized with xylene, and then the slide was immersed in ethanol with decreasing concentrations (100%, 90%, 80%, and 70%). Then, the slide was heated to 90° C. in an antigen recovery solution containing Tris, ethylenediaminetetraacetic acid (EDTA), and Tween 20, and the sample was pre-blocked with FcR antibody (clone: 2.4G2) in PBS containing 3% BSA. The samples were stained overnight at 4° C. in a 1% BSA/PBS with a CD8a antibody. After washing 3 times, the specimen was stained at room temperature for 1 hour with Alexa-488 anti-rat immunoglobulin.

6-4. Tissue Preparation and mIHC

An FFPE tumor tissue slide was dewaxed with xylene, and mIHC staining was performed using a Leica Bond Rx™ Automated Stainer (Leica Biosystems). Then, the specimen was dewaxed with a Leica Bond Dewax solution (Leica Biosystems). Antigen retrieval was performed in a solution having a pH of 9.0, and primary antibody staining was performed using PD-L1 (13684S, Cell signaling, 1:300), FOXP3 (14-5773-82, Invitrogen, 1:100), CD4 (25229S, Cell signaling, 1:100), CD8 (98941S, Cell signaling, 1:200), CD11b (PA5-79533, Invitrogen, 1:150), and CD31 (ab124432, Abcam, 1:150). Bond Epitope Retrieval 1 (Leica Biosystems) was used for antigen retrieval for 20 minutes between all primary antibody staining processes. Opal™ TSA Plus dyes 570, 520, 690, 620, 480, and 780 (Akoya Biosciences) were used for visualization. Subsequently, an Opal™ TSA-DIG reagent (Perkin-Elmer, 1:70) was added to the tissue slide in the last stage of antibody staining to stain nuclei with DAPI. Then, the sample slide was covered with a HIGHDEF® IHC fluoromount (Enzo Life Sciences).

### 6-5. Image Acquisition and Cell Counting

Images were obtained, and cells were counted using a device of PrismCDX (Korea). A PerkinElmer Vectra 3.0 Automated Quantitative Pathology Imaging System (Perkin-Elmer, Waltham, MA) was used for sample scanning. For pure images, emission spectra of both a representative slide and an unstained tissue sample were used. A spectrum library of fluorophores was set by applying the stained sections (CD31-Opal 520, CD11b-Opal 570, PD-L1-Opal 690, FOXP3-Opal 620, CD4-Opal 480, CD8-Opal 780, and DAPI). Immune cell population was identified by InForm image analysis software, and the cells were counted based on detection of DAPI. For co-expression analysis, the IHC scoring method was used, and for this purpose, data obtained using the InForm software was transferred as threshold values for the Spotfire™ software and for positive of each factor. Cells expressing PD-L1, CD11b, Foxp3, CD4, CD8, and CD31 were quantified in each slide, and expression intensity was calculated based on cut-off values and analyzed using the Spotfire™ program.

As a result of identifying in vivo antitumor effects in CT26 cell-bearing mice orally administered with 10 mg/kg OP [ODFS (10)] or SIMVA/DL-CD (20) alone or a combination of SIMVA/DL-CD (20) and ODSF every day for 3 consecutive weeks, administration with SIMVA/DL-CD (20) alone induced a 4.09-fold more decrease in tumor size compared to the control (untreated). Suppression of tumor growth in the mice was improved by the combination of SIMVA/DL-CD (20) and ODSF (10) compared to the administration of SIMVA/DL-CD (20) alone by 15.5 times (FIGS. 10A, and 11B). Meanwhile, a tumor growth suppression rate of ODSF (10) was higher than that of the control by 9.18 times. Also, a tumor growth rate was suppressed by treatment with SIMVA/DL-CD (20)+ODSF (10) more than by treatment with ODSF (10) alone by 6.75 times. Also, the weight of tumors isolated from the group treated with SIMVA/DL-CD (20)+ODSF (10) was lower than that observed after treatment with SIMVA/DL-CD (20) by 825%.

These results clearly indicate that superior therapeutic effects on colorectal cancer were obtained by the combination of SIMVA/DL-CD and ODSF compared to single therapy of each formulation.

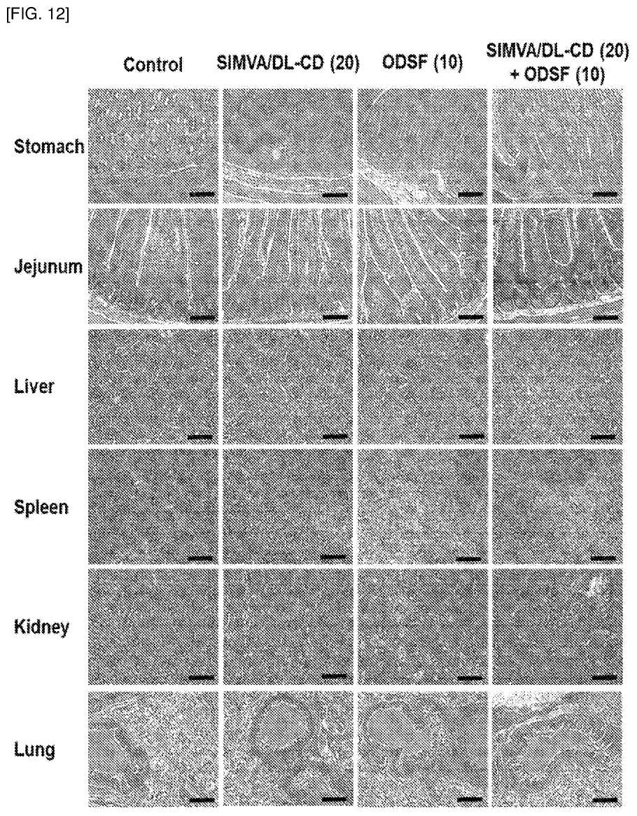

The combination of SIMVA/DL-CD (20)+ODSF (10) caused the weight loss assumed to be due to OP-induced toxicity on day 6 compared to the control or the group treated with SIMVA/DL-CD (20) (FIG. 11A). Although the weight loss lasted up to day 21, toxicity was not observed in stomach, jejunum, liver, spleen, kidney, and lung of all treated groups as a result of hematoxylin and eosin (H&E) staining (FIG. 12).

In order to identify changes in immune cells related to antitumor effects of the combination of SIMVA/DL-CD (20)+ODSF (10), tumor-infiltrating CD8⁺ T cells were subjected to immunohistochemistry on day 18 of treatment. As expected, the combination therapy induced more populations of CD8⁺ T cells than administration of each formulation alone, thereby increasing the number of CD8⁺ T cells in TMEs (FIG. 10B). As a result of additional analysis via multiplex immunohistochemistry (mIHC), the combination therapy increased a density of tumor-infiltrating CD8⁺ T cells compared to the control (FIG. 10C). In addition, the co-administration limited formation of CD11b⁺ cell populations at boundaries of tumors when compared to the control (FIG. 10D). The CD11b⁺ cells were localized with expression of programmed cell death ligand (PD-L1) (FIG. 10E), indicating formation of an immunosuppressing TME capable of impairing an antitumor immune response. In contrast, CD4⁺ T cells and Foxp3⁺CD4⁺ T cells were hardly detected in both TMEs.

Based on these results, it can be seen that combination therapy of SIMVA/DL-CD and ODSF may induce strong antitumor effects by activating CD8⁺ T cell immunity against cancer, which may also be limited by PD-L1-expressing immunosuppressive CD11b⁺ cell populations in the TMEs.

### Example 7: Overcome Resistance to αPD-1 Immune Checkpoint Blockade by SIMVA/DL-CD and ODSF in Colorectal Cancer

Considering a high expression level of programmed death-ligand-1 (PD-L1) weakens CD8⁺ T cell immunity, it was expected that a blockade of programmed cell death protein-1 (PD-1) may improve therapeutic effects of combination therapy of SIMVA/DL-CD and ODSF. Thus, in order to investigate antitumor effects of triple combination therapy using SIMVA/DL-CD, ODSF, and αPD-1, a CT26 murine colon cancer model known to be resistant to immune checkpoint blockade (ICB) was used (FIG. 13A).

Specifically, 1×10⁶ CT26 cells were inoculated on the left side of each male BALB/c mouse. On day 6 after inoculation of the tumor cells, each mouse was administered with αPD-1, SIMVA/DL-CD (20)+ODSF (10), or αPD-1+SIMVA/DL-CD (20)+ODSF (10). All formulations were orally administered once a day, and αPD-1 was intraperitoneally injected. The tumor size was measured every 3 days using a caliper. In order to evaluate immunological memory formation, several mice exhibiting complete tumor suppression in the group treated with αPD-1+SIMVA/DL-CD (20)+ODSF (10) were rechallenged on the opposite side with the same tumor model.

As a result of orally administering SIMVA/DL-CD and ODSF together with αPD-1, antitumor effects were induced (FIG. 13B). Survival of the mice was extended (FIG. 13C), and tumors were completely suppressed in 2 mice among 7 mice (FIG. 13B). Thereafter, as a result of rechallenging the same tumor cell-bearing mice on the opposite side for evaluating immunological memory formation, it was confirmed that tumor growth was suppressed by immunological memory in the tumor model (FIG. 13D).

Based on these results, it can be seen that the combination therapy of SIMVA/DL-CD and ODSF not only sensitizes ICB-resistant tumors but also acts complementarily to αPD-1 therapy to induce a continuous antitumor immune response.

According to the results of the examples described above, solubility and permeability of SIMVA were improved using the SIMVA/DL-CD composite for oral administration according to the present invention using DL as an oral permeation enhancer and Labrasol and P188 as surfactants. In addition, oral absorption of the SIMVA/DL-CD composite was considerably increased, and in vivo experiments for the CT26 tumor-bearing mice administered with the SIMVA/DL-CD composite exhibited considerable tumor growth suppressing effects via elevated anticancer immunity. Particularly, co-administration of the SIMVA/DL-CD composite for oral administration and OP powder induced considerable tumor suppressing effects and CD8⁺ T cell immunity. In addition, the co-administration sensitized tumors resistant to αPD-1 monoclonal antibody to checkpoint blockade, and thus it was confirmed that the SIMVA/DL-CD composite for oral administration according to the present invention has therapeutic potential for effective anticancer immunotherapy.

The above description of the present invention is provided for the purpose of illustration, and it would be understood by those skilled in the art that various changes and modifications may be made without changing the technical conception and essential features of the present invention. Thus, it is clear that the above-described embodiments are illustrative in all aspects and do not limit the present invention. The various embodiments disclosed herein are not intended to be limiting, with the true scope and spirit being indicated by the following claims. The present invention is to be limited only by the terms of the appended claims, along with the full scope of equivalents to which such claims are entitled.

1. A method of preventing or treating a cancer administering a composition to an individual, the composition comprising:
a lipophilic statin;
a composite of deoxycholic acid or a pharmaceutically acceptable salt thereof and an amino acid or an acceptable salt thereof; and
a surfactant.

2. The method of claim 1, wherein the lipophilic statin comprises at least one selected from the group consisting of simvastatin (SIMVA), atorvastatin, lovastatin, fluvastatin, cerivastatin, and pitavastatin.

3. The method of claim 1, wherein the pharmaceutically acceptable salt of deoxycholic acid is selected from the group consisting of a hydrochloride, a sodium salt, a potassium salt, and an ammonium salt.

4. The method of claim 1, wherein the amino acid is a cationic amino acid.

5. The method of claim 4, wherein the cationic amino acid comprises at least one selected from the group consisting of lysine, arginine, and histidine.

6. The method of claim 1, wherein the acceptable salt of the amino acid comprises at least one selected from the group consisting of a hydrochloride, a sodium salt, a potassium salt, and an ammonium salt.

7. The method of claim 1, wherein the composite of deoxycholic acid or a pharmaceutically acceptable salt thereof and an amino acid or an acceptable salt thereof is Na-deoxycholyl-I-lysyl-methylester.

8. The method of claim 1, wherein the surfactant is a non-ionic surfactant.

9. The method of claim 8, wherein the non-ionic surfactant comprises at least one selected from the group consisting of poloxamer, polyvinylpyrrolidone, caprylocaproyl macrogol-8 glyceride (Labrasol), alkyl polysaccharide, Cremophor, glycerol monocaprylocaprate (Capmul MCM), lauroyl macrogol-32 glyceride (Gelucire 44/14), Solutrol, polysorbate (Tween), sorbitan monolaurate (Span), propylene glycol monocaprylate (Capryol 90), propylene glycol dicaprylocaprate (Labrafac PG), oleoryl macrogol-6 glyceride (Labrafil M1944 CS), lauroyl macrogol-6 glyceride (Labrafil M2130 CS), linoleoyl macrogol-6 glyceride (Labrafil M2125 CS), medium-chain triglyceride (Labrafac), oleic acid, stearic acid, glyceryl behenate (Compritol 888), glycerol monostearate, polyethylene glycol, ethoxylate, alkanoamide, amine oxide, acetylene glycol, sugar ester, sorbitan ester, glycerol ester, and glycoside.

10. The method of claim 1, wherein i) the lipophilic statin; ii) the composite of deoxycholic acid or a pharmaceutically acceptable salt thereof and an amino acid or an acceptable salt thereof; and iii) the surfactant constitute a micelle form.

11. The method of claim 1, wherein the composition has improved solubility and permeability of the lipophilic statin.

12. The method of claim 1, wherein the composition has an enhanced in vivo oral bioavailability of the lipophilic statin.

13. The method of claim 1, wherein the composition has antitumor effects.

14. The method of claim 1, wherein the composition is co-administered with an anticancer drug.

15. The method of claim 14, wherein the anticancer drug comprises at least one selected from the group consisting of an alkylating agent, a platinum-based drug, an antimetabolite, an antibiotic, a Taxane-based anticancer drug, a Vinca alkaloid-based anticancer drug, a targeted agent, an immune anticancer drug, a cancer vaccine, a cell therapy product, an oncolytic virus, and any combination thereof.

16. The method of claim 15, wherein the alkylating agent comprises at least one selected from the group consisting of nitrogen mustard-based drugs, ethyleneimine- and methylmelamine-based drugs, methylhydrazine derivatives, alkyl sulfonate-based drugs, nitrosourea-based drugs, and triazine-based drugs.

17. The method of claim 15, wherein the platinum-based drug comprises at least one selected from the group consisting of Cisplatin, Carboplatin, and Oxaliplatin.

18. The method of claim 15, wherein the antimetabolite comprises at least one selected from the group consisting of a folate antagonist-based drug, a purine antagonist-based drug, and a pyrimidine antagonist-based drug.

19. The method of claim 15, wherein the antibiotic comprises at least one selected from the group consisting of Etoposide, Topotecan, Irinotecan, Idarubicin, Epirubicin, Dactinomycin, Doxorubicin, Adriamycin, Daunorubicin, Bleomycin, Mitomycin C, and Mitoxantrone.

20. The method of claim 15, wherein the Taxane-based anticancer drug comprises at least one selected from the group consisting of Paclitaxel and Docetaxel.

21. The method of claim 15, wherein the Vinca alkaloid-based anticancer drug comprises at least one selected from the group consisting of Vincristine, Vinblastine, and Vinorelbine.

22. The method of claim 15, wherein the targeted agent comprises at least one selected from the group consisting of an epidermal growth factor receptor (EGFR) targeted agent, a human epidermal growth factor receptor 2 (HER2) targeted agent, a B cell marker (CD20) targeted agent, a myeloid cell surface antigen (CD33) targeted agent, a cluster of differentiation 52 (CD52) targeted agent, a tumor necrosis factor receptor superfamily member 8 (CD30) targeted agent, a breakpoint cluster region protein-tyrosine-protein kinase (bcr-abl)/tyrosine kinase receptor (c-Kit) targeted agent, an anaplastic lymphoma receptor tyrosine kinase (ALK) targeted agent, an antiangiogenic targeted agent, a mammalian target of rapamycin (mTOR) targeted agent, a cyclin-dependent kinase 4/6 (CDK4/6) targeted agent, a poly (ADP-ribose) polymerase (PARP) targeted agent, a proteasome inhibitor, a tyrosine kinase antagonist, a protein kinase C inhibitor, and a farnesyl transferase inhibitor.

23. The method of claim 15, wherein the immune anticancer drug comprises at least one selected from the group consisting of an anti-programmed cell death protein-1 (PD-1) interaction inhibitor, an anti-programmed cell death ligand-1 (PD-L1) interaction inhibitor, a cytotoxic T lymphocyte associated antigen 4 (CTLA4 or CD152)/B7-1/B7-2 interaction inhibitor, and a cluster of differentiation 47 (CD47)/signal-regulatory protein (SIRP) interaction inhibitor.

24. The method of claim 1, wherein administration of the composition is used in combination with an anticancer therapy.

25. The method of claim 24, wherein the anticancer therapy is selected from the group consisting of radiotherapy and photodynamic therapy.

26. A method of preparing a composition comprising: i) a lipophilic statin; ii) a composite of deoxycholic acid or a pharmaceutically acceptable salt thereof and an amino acid or an acceptable salt thereof; and iii) a surfactant,
the method comprising:
adding the lipophilic statin to an aqueous solution including a first surfactant, followed by stirring to prepare a dispersion;
adding an aqueous solution including the composite of deoxycholic acid or a pharmaceutically acceptable salt thereof and an amino acid or an acceptable salt thereof to the dispersion, followed by stirring to prepare a mixture; and
adding an aqueous solution including a second surfactant to the mixture.

27. The method of claim 26, wherein the composite of deoxycholic acid or a pharmaceutically acceptable salt thereof and an amino acid or an acceptable salt thereof is N^{α}-deoxycholyl-I-lysyl-methylester.

28. The method of claim 26, wherein the first and second surfactants are non-ionic surfactants.

29. The method of claim 28, wherein the non-ionic surfactant comprises at least one selected from the group consisting of poloxamer, polyvinylpyrrolidone, caprylocaproyl macrogol-8 glyceride, alkyl polysaccharide, Cremophor, glycerol monocaprylocaprate, lauroyl macrogol-32 glyceride, Solutrol, polysorbate, sorbitan monolaurate, propylene glycol monocaprylate, propylene glycol dicaprylocaprate, oleoryl macrogol-6 glyceride, lauroyl macrogol-6 glyceride, linoleoyl macrogol-6 glyceride, medium-chain triglyceride, oleic acid, stearic acid, glyceryl behenate, glycerol monostearate, polyethylene glycol, ethoxylate, alkanoamide, amine oxide, acetylene glycol, sugar ester, sorbitan ester, glycerol ester, and glycoside.

30. The method of claim 26, wherein the composition comprising i) the lipophilic statin; ii) the composite of deoxycholic acid or a pharmaceutically acceptable salt thereof and an amino acid or an acceptable salt thereof; and iii) the surfactant constitute a micelle form.

31. (canceled)
